SEQUENCE LISTING (66 sequences)

SEQ ID NO: 1 - DNA, 19 bp, Homo Sapiens
gggatcacactgagcttgc

SEQ ID NO: 2 - DNA, 19 bp, Homo Sapiens
acccctgattctggtgtgg

SEQ ID NO: 3 - DNA, 19 bp, Homo Sapiens
cctggatgcgcaaagttca

SEQ ID NO: 4 - DNA, 22 bp, Homo Sapiens
aattccttctccagctccaaga

SEQ ID NO: 5 - DNA, 20 bp, Homo Sapiens
acggatctataccaacgcca

SEQ ID NO: 6 - DNA, 20 bp, Homo Sapiens
ggggtggaagtcccagtaat

SEQ ID NO: 7 - DNA, 21 bp, Homo Sapiens
cagaacagattggatcggaaa

SEQ ID NO: 8 - DNA, 20 bp, Homo Sapiens
ccgatccaatctgttctggt

SEQ ID NO: 9 - DNA, 23 bp, Homo Sapiens
aataaggcaaaaggaaagcaact

SEQ ID NO: 10 - DNA, 23 bp, Homo Sapiens
caaaacacatcattacacctgct

SEQ ID NO: 11 - DNA, 26 bp, Homo Sapiens
agaccggggttgtcaaaaaactctac

SEQ ID NO: 12 - DNA, 26 bp, Homo Sapiens
tcaggaccacaggcaataaacacatc

SEQ ID NO: 13 - DNA, 19 bp, Homo Sapiens
caagagagcgcagccttag

SEQ ID NO: 14 - DNA, 20 bp, Homo Sapiens
gcaaaagtcagtgctgaacg

SEQ ID NO: 15 - DNA, 22 bp, Homo Sapiens
gagcacagaaagtcatcaaagc

SEQ ID NO: 16 - DNA, 18 bp, Homo Sapiens
tccagaatgtccgccttc

SEQ ID NO: 17 - DNA, 22 bp, Homo Sapiens
caatggaaagggattttaagca

SEQ ID NO: 18 - DNA, 26 bp, Homo Sapiens
tttgagattatgtaccacgttagatg

SEQ ID NO: 19 - DNA, 20 bp, Homo Sapiens
tccaggaacggaaaatcaag

SEQ ID NO: 20 - DNA, 20 bp, Homo Sapiens
gcctcctcatcccctacttc

SEQ ID NO: 21 - DNA, 24 bp, Homo Sapiens
gttattgtgttgccttagcacttc

SEQ ID NO: 22 - DNA, 24 bp, Homo Sapiens
agtgaaatgaattgctcaaattgt

SEQ ID NO: 23 - DNA, 20 bp, Homo Sapiens
cagaccgaagtggaggaaac

SEQ ID NO: 24 - DNA, 21 bp, Homo Sapiens
cctcttccttgtccttctcct

SEQ ID NO: 25 - DNA, 20 bp, Homo Sapiens
acatcatcacagacttggtc

SEQ ID NO: 26 - DNA, 20 bp, Homo Sapiens
cattattgacagcagctgcc

SEQ ID NO: 27 - DNA, 20 bp, Homo Sapiens
ccggcagaagattgtagagc

SEQ ID NO: 28 - DNA, 20 bp, Homo Sapiens
cgttggacacgttttgattg

SEQ ID NO: 29 - DNA, 21 bp, Homo Sapiens
aacacttgaagcccagatgga

SEQ ID NO: 30 - DNA, 20 bp, Homo Sapiens
gcaggctgaattcggttctc

SEQ ID NO: 31 - DNA, 18 bp, Homo Sapiens
gtacccgtcgctgcactc

SEQ ID NO: 32 - DNA, 21 bp, Homo Sapiens
ggcttgaaaggatggctcttg

SEQ ID NO: 33 - DNA, 19 bp, Homo Sapiens
tacaaagttaccagccacc

SEQ ID NO: 34 - DNA, 20 bp, Homo Sapiens
ggaagttgagaggacattga

SEQ ID NO: 35 - DNA, 21 bp, Homo Sapiens
ttggcatgagcagggatatct

SEQ ID NO: 36 - DNA, 20 bp, Homo Sapiens
acggtacaggatgctctcgg

SEQ ID NO: 37 - DNA, 22 bp, Homo Sapiens
gcagaagaaataggagccaatg

SEQ ID NO: 38 - DNA, 22 bp, Homo Sapiens
cgattctctgcagaagatgctc

SEQ ID NO: 39 - DNA, 24 bp, Homo Sapiens
ggctgtcttcatcatcctgctgct

SEQ ID NO: 40 - DNA, 26 bp, Homo Sapiens
gttcttgctctcctgtgcgatcttgt

SEQ ID NO: 41 - DNA, 24 bp, Homo Sapiens
cccctcttcaactttgagaaggga

SEQ ID NO: 42 - DNA, 24 bp, Homo Sapiens
gtgaaggagtatttggggatgcac

SEQ ID NO: 43 - DNA, 20 bp, Homo Sapiens
gctccgagtcttcaagttgg

SEQ ID NO: 44 - DNA, 20 bp, Homo Sapiens
ggttgtttgcatcagggtct

SEQ ID NO: 45 - DNA, 25 bp, Homo Sapiens
caaatctgaaactgcttctgccaca

SEQ ID NO: 46 - DNA, 25 bp, Homo Sapiens
ctagggcccaggggcaatcagctcc

SEQ ID NO: 47 - DNA, 20 bp, Homo Sapiens
cccagcagctgttaaaggag

SEQ ID NO: 48 - DNA, 20 bp, Homo Sapiens
ctgggacagtcgtttggttt

SEQ ID NO: 49 - DNA, 21 bp, Homo Sapiens
cagcgctggaggtggatattc

SEQ ID NO: 50 - DNA, 21 bp, Homo Sapiens
cacacacagtggcttggactc

SEQ ID NO: 51 - DNA, 20 bp, Homo Sapiens
ggcagaggtctctgttcagg

SEQ ID NO: 52 - DNA, 20 bp, Homo Sapiens
tgaactcgaaagggttgtcc

SEQ ID NO: 53 - DNA, 20 bp, Homo Sapiens
gtggcggaaaatcttgtagg

SEQ ID NO: 54 - DNA, 20 bp, Homo Sapiens
ccccattgttcatgtgagtg

SEQ ID NO: 55 - DNA, 20 bp, Homo Sapiens
caactgcagctgtgacatcc

SEQ ID NO: 56 - DNA, 20 bp, Homo Sapiens
gcccagtgactatccagtcc

SEQ ID NO: 57 - DNA, 20 bp, Homo Sapiens
ggtgaacacagacgctatgc

SEQ ID NO: 58 - DNA, 20 bp, Homo Sapiens
tctcccacttggtgttcctc

SEQ ID NO: 59 - DNA, 20 bp, Homo Sapiens
tgcactggtgcaggactatg

SEQ ID NO: 60 - DNA, 20 bp, Homo Sapiens
aaggctttggaacccacatt

SEQ ID NO: 61 - DNA, 20 bp, Homo Sapiens
acgacacccaccacctacat

SEQ ID NO: 62 - DNA, 20 bp, Homo Sapiens
tacagataggcagggcgttc

SEQ ID NO: 63 - DNA, 20 bp, Homo Sapiens
gaaccacggcactgattttc

SEQ ID NO: 64 - DNA, 20 bp, Homo Sapiens
cacagctccccaccatattc

SEQ ID NO: 65 - DNA, 8 bp, Homo Sapiens
  misc_feature (1)..(1): n is a, c, g, or t
  misc_feature (6)..(8): n is a, c, g, or t
ntgcannn

SEQ ID NO: 66 - DNA, 7 bp, Homo Sapiens
tttgcat

(19) United States
(12) Patent Application Publication
(10) Pub. No.: US 2018/0148687 A1
(43) Pub. Date: May 31, 2018

(54) GENERATING INDUCED NEURAL PROGENITOR CELLS FROM BLOOD

(71) Applicant: McMaster University, Hamilton (CA)

(72) Inventors: Mickie Bhatia, Ancaster (CA); Jong-Hee Lee, Ancaster (CA); Ryan Mitchell, Mount Hope (CA); Tony Collins, Hamilton (CA)

(21) Appl. No.: 15/575,029

(22) PCT Filed: May 19, 2016
(86) PCT No.: PCT/CA2016/050566

§ 371 Date: Nov. 17, 2017

Related U.S. Application Data
(60) Provisional application No. 62/164,222, filed on May 20, 2015.

(51) Int. Cl. C12N 5/0793; G01N 33/50

(52) U.S. Cl. CPC C12N 5/0619; G01N 33/5058; C12N 2506/11; C12N 2501/71; C12N 2501/727; C12N 2501/72; C12N 2501/2303; C12N 2501/50

(57) ABSTRACT

The present disclosure provides a method of generating induced neural progenitor cells from CD34+/CD45+ blood cells using a POU domain containing gene or protein and inhibitors of Smad and GSK-3β, without traversing the pluripotent state. Also provided are uses and assays of the cells produced by the methods of the disclosure.

### CROSS-REFERENCE TO RELATED APPLICATIONS

This application claims the benefit of priority to U.S. Provisional Application No. 62/164,222 filed May 20, 2015, the contents of which is incorporated herein by reference in its entirety.

### FIELD

The disclosure relates to reprogramming of blood cells. In particular, the disclosure relates to methods of generating induced neural progenitor cells derived from CD34⁺/CD45⁺ blood cells.

### BACKGROUND

The reprogramming of adult cells into alternative tissues holds promise for regenerative medicine and drug discovery, especially for human cell types that are difficult to procure such as neural tissue (Sancho-Martinez et al., 2012). However, significant limitations remain using current technology as it relates to human sources, thus novel approaches that allow generation of large numbers of renewable neural cells from easily accessible tissues derived from donors is required. Complete cellular reprogramming to the pluripotent state has gone some way to realize this promise (Takahashi and Yamanaka, 2006). However, although transformative, this advance is limited by costly and time consuming methods over several months to first derive skin fibroblasts and then generate and characterize resulting iPSCs (Stacey et al., 2013). Furthermore, resulting iPSCs acquire inefficiencies in lineage specific differentiation from pluripotent state that limits reproducible production of specific mature cell types (Lee et al., 2014). Similarly, use of hiPSCs in cell replacement therapy continues to precipitate barriers and concerns that require laborious measures to assure resulting cells are free from tumor forming pluripotent cells has yet to be resolved (Cunningham et al., 2012).

More recent studies have established a paradigm whereby forced expression of lineage-specific factors allows direct reprogramming into differentiated somatic cells, including cardiomyocytes, hepatocyte-like cells, blood and neurons without iPSC formation (Efe et al., 2011; Pang et al., 2011; Szabo et al., 2010). However, direct cell fate reprogramming of human cells is accompanied by other limitations and remains inefficient, requiring multiple transcription factors to be ectopically expressed in every cell, and is largely based on difficult to obtain human skin biopsies that are not available from historical clinical studies. Alternatively, blood cells can be readily obtained from patients, require no culture derivation prior to reprogramming, and have been stored and banked (Broxmeyer, 2010) from large cohort patient trials in the past such as those suffering from neurological disorders (http://brainbank.ucla.edu; http://www.clsa-elcv.ca/).

### SUMMARY

The present inventors have shown that OCT4 induced plasticity reprogramming combined with neural potentiating small molecules directly converts human blood progenitors derived from both cord blood and adult sources to neural progenitor cells (NPCs). The present inventors further demonstrate that these human Blood derived (BD) NPCs are capable of in vivo differentiation and survival as well as tri-potent neural differentiation in vitro that includes neuronal differentiation towards clinically relevant CNS and PNS subtypes.

Accordingly, the present disclosure provides a method of generating induced neural progenitor cells from CD34+/CD45⁺ blood cells comprising:

a) providing CD34⁺/CD45⁺ blood cells that ectopically express, overexpress or are treated with a POU domain containing gene or protein and culturing said cells media to allow expression of the POU domain containing gene or protein; and

b) culturing the cells produced in (a) in basal neural progenitor media supplemented with inhibitors of Smad and GSK-3β to produce induced neural progenitor cells;

wherein induced neural progenitor cells are generated without traversing the pluripotent state.

In an embodiment, the cells in (a) are cultured in hematopoietic stem cell culture media followed by reprogramming media to allow expression of the POU domain containing gene or protein.

In one embodiment, the method further comprises after (b) maintaining the cells produced in (b) in neural induction media for growing or expanding the induced neural progenitor cells.

In an embodiment, CD34⁺/CD45⁺ blood cells that ectopically express a POU domain containing gene or protein in (a) are produced by lentiviral transduction. In an embodiment, the lentiviral transduction occurs in hematopoietic stem cell culture media and then the cells are transferred to reprogramming media and cultured prior to step (b).

In another embodiment, the CD34⁺/CD45⁺ blood cells that are treated with a POU domain containing gene or protein in (a) are produced by providing an exogenous POU domain containing gene or protein.

The POU domain containing gene or protein is an Oct gene or protein, such as Oct-1, -2, -4 or -11. In one embodiment, the Oct gene or protein is Oct-4.

In an embodiment, the CD34⁺/CD45⁺ blood cells are derived from peripheral blood. In another embodiment, the CD34⁺/CD45⁺ blood cells are derived from umbilical cord blood.

The cells in (a) are optionally cultured in the hematopoietic stem cell culture media for 2-4 days. In one embodiment, the hematopoietic stem cell culture media comprises SCF, Flt-3L, IL-3 and/or TPO. In an embodiment, the hematopoietic stem cell culture media comprises SCF, Flt-3L, IL-3 and TPO.

The cells in (b) are optionally cultured in reprogramming media for 4-7 days. In one embodiment, the reprogramming media comprises bFGF. In another embodiment, the reprogramming media comprises DMEM/F12, 20% Knockout Serum Replacement and bFGF.

The inhibitors of Smad are compounds that inhibit Smad signaling. In one embodiment, the Smad inhibitors comprise at least one of SB431542, LDN-193189, and Noggin. The inhibitors of GSK-3β are compounds that inhibit GSK-3β signaling. In one embodiment, the GSK-3β inhibitor is CHIR99021. In an embodiment, the inhibitors of Smad and GSK-3β of (c) comprise SB431542, LDN-193189, Noggin and CHIR99021.

The cells in (c) are optionally cultured in the basal neural progenitor media supplemented with the inhibitors of Smad and GSK-3β for 10-14 days.

In an embodiment, the neural induction media comprises basal neural progenitor media supplemented with bFGF and EGF.

In a further embodiment, the methods disclosed herein further comprise culturing the cells produced in (d) in differentiation medium under conditions that allow production of differentiated cells. In an embodiment, the differentiated cells are neurons, optionally sensory neurons. In another embodiment, the differentiated cells are glial cells, optionally astrocytes or oligodendrocytes.

Also provided herein are isolated progenitor or differentiated cells generated by the methods disclosed herein.

Even further provided is a use of the cells generated by the methods disclosed herein for engraftment or cell replacement in a subject in need thereof, optionally for autologous or non-autologous transplantation in a subject in need thereof. In an embodiment, the subject is a human.

Also provided herein is a method of screening progenitor cells or cells derived therefrom comprising
a) preparing a culture of progenitor or differentiated cells by the methods disclosed herein;
b) treating the cells with a test agent or agents; and
c) subjecting the cells to analysis.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows generation of iNPC from neonatal and adult blood cells. (A) Schematic for deriving iNPCs from lineage depleted CD34⁺CD45⁺ blood. (B) Phase-contrast images of iNPCs, bar=300 μm. (C) iNPC colony numeration (bar=Std Dev). (D) iNPC colony numeration from CD34⁺ or CD34⁻ cells (bar=Std Dev). (E) Phase-contrast image of iNPC spheres, bar=300 μm. (F) Immunofluorescence of iNPCs for PAX6, Nestin and SOX2, bar=100 μm. (G) FACS for PAX6 and NESTIN, from iNPCs (n=4). (H) Predicted iNPCs from 50K human blood progenitors (bar=Std Dev).

FIG. 2 shows molecular profiling of OCT4 BD-iNPC generation. (A) Hierarchal cluster analysis on global gene expression of Fib-iNPC and BD-iNPC+/−inhibitors with primary human NPC. (B) Number of genes changing in response to inhibitors in Fib-iNPC versus hBD-iNPCOCT4 (false discovery rate (FDR) p≤0.05, fold change≥1.5). (C) Gene set enrichment analysis (GSEA) on iNPC+/−inhibitors. (D) Expression of hematopoietic or neural specific genes in BD-iNPCs and control NPCs derived from hPSCs (bar=Std Dev).

FIG. 3 shows in vivo and in vitro differentiation potential of BD-iNPCs. (A) Montage of individual images from sectioned brain tissue 3 weeks after injection of BD-iNPCs expressing GFP (“R” zoomed images have been manually aligned for visual continuity). (B) In vivo differentiation of BD-iNPCs into neurons (expression of Tuj1, MAP2 and NeuN) and astrocytes (expression of GFAP). C-E. In vitro differentiation of BD-iNPCs in to GFAP-positive astrocytes (C) and 04-positive oligodendrocytes (D). (E) Tuj1 and MAP2 positive neurons and glutamatergic and GABAergic neuronal subtypes. bar=100 μm. (F) Expression of Synapsin, scale bar=50 μm. (G) Raw traces of membrane potential changes to stepwise current injection of equal increment. (H) Repetitive action potential firing was induced upon depolarizing current injection. (I) TTX-sensitive fast inward currents on depolarization. (J) TH and Nurr1 positive dopaminergic (DA) neurons derived from BD-iNPCs. bar=100 μm. (K) HPLC for dopamine (left) and levels in multiple DA cultures (right) (bar=Std Dev). (L) Gene set enrichment analysis for LEE_NEURAL_CREST_STEM_CELL gene list between human fibroblasts and human blood. (M) Gene set enrichment analysis for LEE_NEURAL_CREST_STEM_CELL gene list between human blood and BD-iNPCs.

FIG. 4 shows generation of functional nociceptive neurons that model chemotherapy induced neuropathy. (A) Tuj1⁺ neurons express BRN3A and ISL1. bar=50 μm. (B) Expression of NTRK1 by FACS. (C) Transcript expression of BRN3A, ISL1 and NTRK1 during differentiation from iNPCs toward sensory neurons. (D) Neuronal clustering (top left) and Substance P expression (low left). bar=100 μm. (right) RT-PCR for TAC1 (Substance P). (E) Expression of channels, channel subunits and receptors, specific to nociceptors. (F) Immunocytochemistry for P2X₃. bar=100 μm. (G, H) Calcium flux in response to 30 μM α,β-methylene ATP treatment of day-14 sensory neurons derived from adult PB-iNPCs. (I) Calcium trace and (J) distribution of cells responsive to 30 μM α,β-methylene ATP and 1 μM capsaicin. (K) P2X₃ antagonist A-317491 significantly inhibited the calcium-response to α,β-methylene ATP (L) Predicted # of nociceptors from 50K human blood progenitors. (M) In vitro response of BD-iNPC derived sensory neurons 48 Hr post Taxol treatment (left) and normalized dose-dependent neurite length and cell count (right). Where applicable (bar=Std Dev).

FIG. 5 shows generation of iNPC from neonatal and adult blood cells related to FIG. 1. (A) Preparation of CD34 CD45⁺ blood cells from lineage depleted mononuclear cells from adult peripheral blood or umbilical cord blood shown in representative panel. (B) FACS analysis shows CD34⁺CD45⁺ human blood cells are devoid of pluripotent markers (SSEA3, TRA1-60), early neural markers (Nestin, PAX6) and neural crest (NC) markers (p75, CD57). (C) Upon exogenous expression of Oct4, along with inhibition of SMAD and GSK-3β, human CD34⁺CD45⁺ blood cells acquire neuronal marker, Nestin as shown in representative flow cytometry plot. (D) Human BD-putative iNPCs do not express hallmark pluripotent markers, SSEA3 and TRA1-60 as compared to hPSCs, suggesting iNPCs generated bypass pluripotent states as measured by phenotypic alterations. (E) In vivo transplantation of BD-iNPCs fail to generate teratomas upon intratesticular injection into NOD/SCID recipients (top). As a positive control, hiPSCs lines were used and induced teratoma formation (bottom). (F) Immunofluorescence analysis of iNPCs with antibodies to neural stem cell marker, CD133. Expression of Ki67 indicated proliferation property of iNPCs. (G) Phase contrast image of H9 derived NPCs. (H-J) Stable expansion of human blood derived iNPCs over long-term passages in vitro. (H) Flow cytometric analysis showed that BD-iNPCs stably expressed makers associated with adult human neural stem cells, PAX6 and Nestin after from 6 passages up to long-term in vitro expansion up to 30 passages (p30). (I) Quantification of flow analysis represented in H. (J) Comparative analyses of expression of genes associated with neural lineage development over long-term culture from p6 to p30.

FIG. 6 shows genomic integrity of BD-iNPCs and loss of hematopoietic and gain of neural transcriptional programming during BD-iNPC generation related to FIG. 2. Array Comparative Genomic Hybridization (aCGH) analysis of 2 different clones of NPCs (A and B) in the early passage was compared to late passage number and no statistically significant (minimum genomic markers of 10 to specify genomic region and p-value<0.001) chromosomal aberrations were found. Tissue Expression analysis on DAVID Bioinformatics Resource tool shows enrichment of up-regulated genes within neural programs and down-regulated genes within hematopoietic programs (C).

FIG. 7 shows in vivo and in vitro differentiation potential of BD-iNPCs related to FIGS. 3 and 4. A-H. Effects of small molecule inhibitors in the self-renewal and developmental potential of human BD-iNPCs. (A) Quantitative analysis of BD-iNPCs expansion in the presence or absence of inhibitors. (B) Schematic strategy for neuronal differentiation of BD-iNPCs cultured in the presence or absence of small molecule inhibitors. (C) Immunofluorescence analysis with antibodies to neuron marker Tuj1, and to neural stem cell marker, PAX6. (D) Quantitative analysis of representative images shown in C. (E) Schematic strategy for differentiating BD-iNPCs into neurons after removing inhibitors in the cultures. (F) Immunofluorescence analysis with antibodies to Tuj1 and PAX6, after culturing BD-iNPCs based on schematic strategy shown in E. (G) Intracellular analysis by flow cytometry for PAX6 from BD-iNPCs cultured in the presence or absence of small molecule inhibitors with EGF and bFGF. (H) Frequency of PAX6 positive cells (left) and mean fluorescent intensity (MFI) (right) from flow cytometry analysis shown in G for human BD-iNPCs. (I) GFAP-positive astrocytes (left), 04-positive oligodendrocytes (middle), and Tuj1 positive neurons (right) derived from hPSCs. (J) Tuj1 and MAP2 positive neurons derived from adult BD-iNPCs. (K) Expression of residual exogenous OCT4 in established hiNPCs and silencing in mature neurons. GAPDH used as control. (L) Voltage-clamp recordings reveal both fast inactivating inward and outward currents indicating functional voltage-dependent Na+ and K+ channels.

FIG. 8 shows generation of functional nociceptive neurons that model chemotherapy induced neuropathy related to FIG. 4. (A) Scheme of protocol used to generate nociceptive sensory neuronal development from human BD-iNPCs. (B) Differentiated nociceptive sensory neuron shows high levels of glutamate, consistent with an excitatory glutamatergic neuron. (C) RT-PCR for sensory neuron marker genes (D) Photomontage of calcium flux images of neurons derived from BD-iNPC calcium response at day 7 (left) and day 14 (right) upon treatment with 30 μM α,β-methylene-ATP or 1 μM capsaicin. The calcium ionophore ionomycin was used as a dye loading control.

### DETAILED DESCRIPTION

Accordingly, the present disclosure provides a method of generating induced neural progenitor cells from CD34⁺/CD45⁺ blood cells comprising:

a) providing CD34⁺/CD45⁺ blood cells that ectopically express, overexpress or are treated with a POU domain containing gene or protein and culturing said cells in media to allow to allow expression of the POU domain containing gene or protein;

b) culturing the cells produced in (a) in basal neural progenitor media supplemented with inhibitors of Smad and GSK-3β to produce induced neural progenitor cells;

wherein induced neural progenitor cells are generated without traversing the pluripotent state.

In an embodiment, the cells in (a) are cultured in hematopoietic stem cell culture media followed by reprogramming media to allow expression of the POU domain containing gene or protein.

In one embodiment, the method further comprises after (b) maintaining the cells produced in (b) in neural induction media for growing or expanding the induced neural progenitor cells.

The term “POU domain containing gene or protein” as used herein refers to a gene or protein containing a POU domain that binds to Octamer DNA binding sequences, such as ntgcannn (SEQ ID NO:65, wherein n is a, c, g, or t, for example, the sequence tttgcat (SEQ ID NO:66). In one embodiment, the POU domain containing gene or protein is an Oct gene or protein, including without limitation, the Oct-1, -2, -4, or -11. In a particular embodiment, the Oct gene or protein is Oct-4.

The term “progenitor cell” as used herein refers to a less specialized cell that has the ability to differentiate into a more specialized cell.

The phrase “without traversing the pluripotent state” as used herein refers to the direct conversion of the CD34⁺/CD45⁺ blood cell to the neural progenitor cell, for example, the produced cells lack pluripotent stem cell properties, such as Tra-1-60 or SSEA3. In an embodiment, the cells do not form teratomas.

The term “CD34⁺/CD45⁺ blood cell” as used herein refers to a hematopoietic progenitor cell that displays the CD34 and CD45 glycoproteins on its cell surface. CD34 is a glycosylated transmembrane protein and represents a well-known marker for primitive blood- and bone marrow-derived progenitor cells, especially for hematopoietic and endothelial stem cells. CD45 is a protein phosphatase glycoprotein expressed in all nucleated hematopoietic cells. In an embodiment, the CD34⁺/CD45⁺ blood cells are derived from peripheral blood. In another embodiment, the CD34⁺/CD45⁺ blood cells are derived from umbilical cord blood.

Methods of obtaining CD34⁺/CD45⁺ blood cells are known in the art. In brief, Mononuclear cells may be isolated by using density gradient centrifugation. CD34⁺/CD45⁺ cells were selected by using an immunomagnetic separation system (Miltenyi Biotec).

The terms “neural progenitor cell” or “induced neural progenitor cell” are used herein interchangeably to refer to a cell that gives rise to cells of the neural lineage, including, without limitation, neurons and glial cells, for example, astrocytes and oligodendrocytes. Neural progenitor markers include, without limitation, A2B5, nestin, PAX6, Sox2, CD133, GFAP, beta tubulin III, and tyrosine Hydroxylase. In an optional embodiment, the neural cells are sorted using these markers.

The term “Oct-4” as used herein refers to the gene product of the Oct-4 gene and includes Oct-4 from any species or source and includes analogs and fragments or portions of Oct-4 that retain enhancing activity. The Oct-4 protein may have any of the known published sequences for Oct-4 which can be obtained from public sources such as Genbank. An example of such a sequence includes, but is not limited to, NM_002701. OCT-4 also referred to as POU5-F1 or MGC22487 or OCT3 or OCT4 or OTF3 or OTF4.

The term “Oct-1” as used herein refers to the gene product of the Oct-1 gene and includes Oct-1 from any species or source and includes analogs and fragments or portions of Oct-1 that retain enhancing activity. The Oct-1 protein may have any of the known published sequences for Oct-1 which can be obtained from public sources such as Genbank. An example of such a sequence includes, but is not limited to, NM_002697.2. Oct-1 also referred to as POU2-F1 or OCT1 or OTF1.

The term “Oct-2” as used herein refers to the gene product of the Oct-2 gene and includes Oct-2 from any species or source and includes analogs and fragments or portions of Oct-2 that retain enhancing activity. The Oct-2 protein may have any of the known published sequences for Oct-2 which can be obtained from public sources such as Genbank. An example of such a sequence includes, but is not limited to, NM_002698.2. Oct-2 is also referred to as POU2-F2 or OTF2.

The term “Oct-11” as used herein refers to the gene product of the Oct-11 gene and includes Oct-11 from any species or source and includes analogs and fragments or portions of Oct-11 that retain enhancing activity. The Oct-11 protein may have any of the known published sequences for Oct-11 which can be obtained from public sources such as Genbank. An example of such a sequence includes, but is not limited to, NM_014352.2. Oct-11 is also referred to as POU2F3.

In one embodiment, CD34⁺/CD45⁺ blood cells that express a POU domain containing gene or protein, such as Oct-1, -2, -4 or -11, include overexpression of the endogenous POU domain containing gene or ectopic expression of the POU domain containing gene or protein. In an embodiment, the CD34⁺/CD45⁺ blood cells do not additionally overexpress or ectopically express or are not treated with other transcription factors, such as Sox2.

CD34⁺/CD45⁺ blood cells that express a POU domain containing protein or gene, such as Oct-1, -2, -4 or -11, can be obtained by various methods known in the art, including, without limitation, by overexpressing endogenous POU domain containing gene, or by introducing a POU domain containing protein or gene into the cells to produce transformed, transfected or transduced cells. The terms “transformed”, “transfected” or “transduced” are intended to encompass introduction of a nucleic acid (e.g. a vector) into a cell by one of many possible techniques known in the art. For example, nucleic acid can be introduced into mammalian cells via conventional techniques such as calcium phosphate or calcium chloride co-precipitation, DEAE-dextran mediated transfection, lipofectamine, electroporation or microinjection or via viral transduction or transfection. Suitable methods for transforming, transducing and transfecting cells can be found in Sambrook et al. (Molecular Cloning: A Laboratory Manual, 3rd Edition, Cold Spring Harbor Laboratory Press, 2001), and other laboratory textbooks. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

In one embodiment, CD34⁺/CD45⁺ blood cells that express a POU domain containing gene or protein or functional variants or fragments thereof are produced by lentiviral transduction. In an embodiment, the lentiviral transduction occurs in hematopoietic stem cell culture media and then the cells are transferred to reprogramming media and cultured prior to step (b).

In another embodiment, the CD34⁺/CD45⁺ blood cells that are treated with a POU domain containing gene or protein include addition of exogenous POU domain containing protein or functional variants or fragments thereof or peptide mimetics thereof. In another embodiment, the CD34⁺/CD45⁺ blood cells that are treated with a POU domain containing gene or protein include addition of a chemical replacer that can be used that induces a POU domain containing gene or protein expression.

The POU domain containing proteins may also contain or be used to obtain or design “peptide mimetics”. For example, a peptide mimetic may be made to mimic the function of a POU domain containing protein. “Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features. Peptide mimetics also include molecules incorporating peptides into larger molecules with other functional elements (e.g., as described in WO 99/25044). Peptide mimetics also include peptoids, oligopeptoids (Simon et al (1972) Proc. Natl. Acad, Sci USA 89:9367) and peptide libraries containing peptides of a designed length representing all possible sequences of amino acids corresponding to a POU domain containing peptide.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of the secondary structures of the proteins described herein. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

The term “variant” as used herein includes modifications, substitutions, additions, derivatives, analogs, fragments or chemical equivalents of the POU domain containing proteins that perform substantially the same function in substantially the same way. For instance, the variants of the POU domain containing proteins would have the same function of being useful in binding the Octamer sequences disclosed herein.

The term “Smad” as used herein refers to proteins in the signaling pathway downstream of TGF-beta binding to its receptor and inhibitors of Smad refer to compounds that inhibit such signaling.

The term “GSK-3β” or “glycogen synthase kinase-beta 3 (NM_001146156)” as used herein refers to a proline-directed serine-threonine kinase that was initially identified as a phosphorylating and an inactivating agent of glycogen synthase and inhibitors of GSK-3β refer to compounds that inhibit the kinase activity.

The term “inhibitor” as used herein refers to any substance that is capable of inhibiting the Smad signaling pathway and/or GSK-3β kinase activity. Such inhibitors optionally include antisense nucleic acid molecules, proteins, antibodies (and fragments thereof), small molecule inhibitors and other substances.

The inhibitors of Smad are compounds that inhibit Smad signaling. In one embodiment, the Smad inhibitors comprise at least one of SB431542 (CAS No: 301836-41-9) (Table 3), LDN-193189 (CAS No: 1062368-24-4) (Table 3), and Noggin (Genbank Accession: NM_005458). The inhibitors of GSK-3β are compounds that inhibit GSK-3β kinase activity. In one embodiment, the GSK-3β inhibitor is CHIR99021 (CAS No: 252917-06-9) (Table 3). In an embodiment, the inhibitors of Smad and GSK-3β used in the methods described herein comprise SB431542, LDN-193189, Noggin and CHIR99021. SB431542 is a selective transforming growth factor-beta (TGF-beta) receptor inhibitor, other known inhibitors include, without limitation: A 83-01, D 4476, GW 788388, LY 364947, R 268712, RepSox, SB 505124, SB 525334 and SD 208. LDN-193189 is a bone morphogenic protein (BMP) receptor inhibitor, other known inhibitors include, without limitation: DMH-1, Dorsomorphin dihydrochloride, K 02288, and ML 347. CHIR99021 is a GSK-3 inhibitor, other known inhibitors include, without limitation: 3F8, A 1070722, AR-A 014418, BIO, BIO-acetoxime, L803-mts, SB 216763, SB 415286, TC-G 24, TCS 2002, and TWS 119. Accordingly, in other embodiment, one or more of the other known inhibitors of Smad and GSK-3β are used in the methods disclosed herein.

Hematopoietic stem cell culture media and conditions for culturing said cells are known in the art. Such media supports growth of hematopoietic stem cells. In one embodiment, the hematopoietic stem cell culture medium comprises at least one hematopoietic cytokine, such as Flt3, SCF, IL-3, or TPO. In one embodiment, the hematopoietic stem cell culture media comprises SCF, Flt-3L, IL-3 and TPO. In an embodiment, the cells in (a) are cultured in hematopoietic stem cell culture media for 2-4 days.

Reprogramming media and conditions for culture are known in the art. In one embodiment, the cells in (a) are cultured in reprogramming media supplemented with bFGF. In another embodiment, the reprogramming media comprises DMEM/F12, 20% Knockout Serum Replacement and is supplemented with bFGF. In an embodiment, the cells are cultured in reprogramming media for 4-7 days. In an embodiment, the cells in (a) are first cultured in hematopoietic stem cell culture media and then cultured in reprogramming media.

Basal neural progenitor media is known in the art and supports growth of neural cells. In an embodiment, the basal media comprises DMEM/F12, 1×N2 and 1×B27. In one embodiment, the cells in (c) are optionally cultured in the basal neural progenitor media comprising the inhibitors of Smad and GSK-3β for 10-14 days.

Neural induction media is known in the art and supports the maintenance of neural progenitor cells. In one embodiment, the neural induction media comprises basal neural progenitor media supplemented with bFGF and EGF.

In a further embodiment, the methods disclosed herein further comprise culturing the cells produced by the methods disclosed herein in differentiation medium under conditions that allow production of differentiated cells. Such conditions are known in the art. See for example, the materials and methods disclosed herein. In an embodiment, the differentiated cells are neurons, optionally GABA neurons, DA neurons and nociceptive sensory neurons. In another embodiment, the differentiated cells are glial cells, optionally astrocytes or oligodendrocytes.

In another aspect, the present disclosure provides isolated progenitor or differentiated cells generated by the methods described herein. Such cells do not express a number of pluripotency markers, such as TRA-1-60 or SSEA-3.

In yet another aspect, the disclosure provides use of the cells described herein for engraftment or cell replacement. In another embodiment, the disclosure provides the cells described herein for use in engraftment or cell replacement. Further provided herein is use of the cells described herein in the manufacture of a medicament for engraftment or cell replacement. “Engraftment” as used herein refers to the transfer of the induced neural progenitor cells produced by the methods described herein to a subject in need thereof. The graft may be allogeneic, where the cells from one subject are transferred to another subject; xenogeneic, where the cells from a foreign species are transferred to a subject; syngeneic, where the cells are from a genetically identical donor or an autograft, where the cells are transferred from one site to another site on the same subject. Accordingly, also provided herein is a method of engraftment or cell replacement comprising transferring the cells described herein to a subject in need thereof. The term “cell replacement” as used herein refers to replacing cells of a subject, such as neurons or glial cells or neural progenitors. In yet another embodiment, cells for engraftment or cell replacement may be modified genetically or otherwise for the correction of disease. CD34⁺/CD45⁺ blood cells before or after transfection or transduction with a POU domain containing gene may be genetically modified to overexpress a gene of interest capable of correcting an abnormal phenotype, cells would be then selected and transplanted into a subject. In another aspect, CD34⁺/CD45⁺ blood cells or POU domain containing gene-expressing CD34⁺/CD45⁺ blood cells overexpressing or lacking complete expression of a gene that is characteristic of a certain disease would produce neural progenitor or differentiated cells for disease modeling, for example drug screening.

The term “subject” includes all members of the animal kingdom, including human. In one embodiment, the subject is an animal. In another embodiment, the subject is a human.

In one embodiment, the engraftment or cell replacement described herein is for autologous or non-autologous transplantation. The term “autologous transplantation” as used herein refers to providing CD34⁺/CD45⁺ blood cells from a subject, generating neural progenitor or differentiated cells from the isolated CD34⁺/CD45⁺ blood cells by the methods described herein and transferring the generated neural progenitor or differentiated cells back into the same subject. The term “non-autologous transplantation” refers to providing CD34⁺/CD45⁺ blood cells from a subject, generating neural progenitor or differentiated cells from the isolated CD34⁺/CD45⁺ blood cells by the methods described herein and transferring the generated neural progenitor or differentiated cells back into a different subject.

In yet another aspect, the disclosure provides use of the cells described herein as a source of neural cells. Such sources can be used for replacement, research and/or drug discovery.

The methods and cells described herein may be used for the study of the cellular and molecular biology of neural progenitor cell development, for the discovery of genes, growth factors, and differentiation factors that play a role in differentiation and for drug discovery. Accordingly, also provided herein is a method of screening progenitor cells or cells derived therefrom comprising
a) preparing a culture of progenitor or differentiated cells by the methods disclosed herein;
b) treating the cells with a test agent or agents; and
c) subjecting the cells to analysis.

In one embodiment, the test agent is a chemical or other substance, such as a drug, being tested for its effect on the differentiation of the cells into specific cell types. In such an embodiment, the analysis may comprise detecting markers of differentiated cell types. For example: for neural differentiation: beta III tubulin, MAP2, GFAP, Oligo4, Glutamate, GABA, tyrosin hydroxylase, Nurr1, Synapsin); for neural precursors PAX6, SOX2, Nestin, CD133; for sensory neurons BRN3A, ISL1, NTRK1, P2x3, and Substance P. In another embodiment, the test agent is a chemical or drug and the screening is used as a primary or secondary screen to assess the efficacy and safety of the agent. Such analysis can include measuring cell proliferation or death or cellular specific features such as Neural signaling, presence of action potential, secretion of certain proteins, activation of specific genes or proteins, activation or inhibition of certain signaling cascades, calcium signaling, and neurite length.

Also provided herein is a method of screening for a compound that modulates the activity, function, viability and/or morphology of sensory neurons comprising:
a) preparing a culture of sensory neurons by the methods disclosed herein;
b) treating the cells with a test compound; and
c) testing the cells for a compound that modulates the activity, function, viability and/or morphology compared to a control in the absence of test compound.

In one embodiment, the test compound is screened for the effect of decreasing or increasing viability of sensory neuron cells compared to control. In another embodiment, the test compound is screened for the effect of decreasing or increasing neurite length of the sensory neuron cells compared to control. In an embodiment, identification of a test compound as capable of increasing viability or neurite strength indicates that the compound is a candidate for treating neuropathies, such as diabetic-induced neuropathy.

In another embodiment, the test compound is screened for the effect of causing neuropathy. In such an embodiment, the compound may be a candidate for anti-cancer treatment. In another embodiment, the test compound is screened in the presence of a chemotherapeutic agent that is known to cause neuropathy and the effect of the test compound in alleviating the neuropathy compared to control is measured.

In yet another embodiment, the test compound is screened for the effect of changes in calcium mobilization.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### Examples

### Results

Generation of iNPCs from Neonatal and Adult Blood Cells Using OCT4 and SMAD+GSK-3 Inhibition

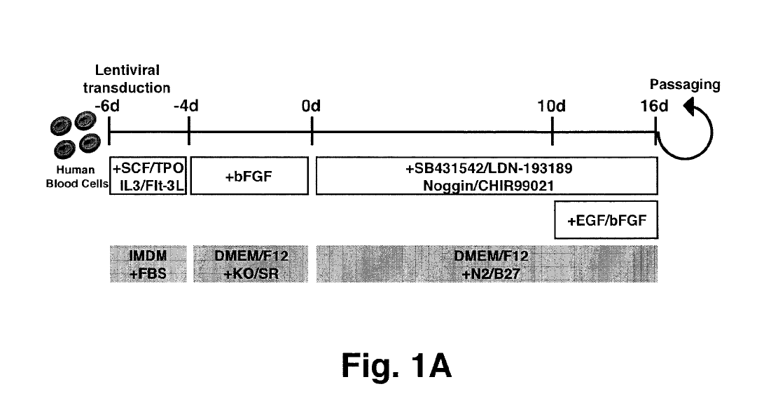

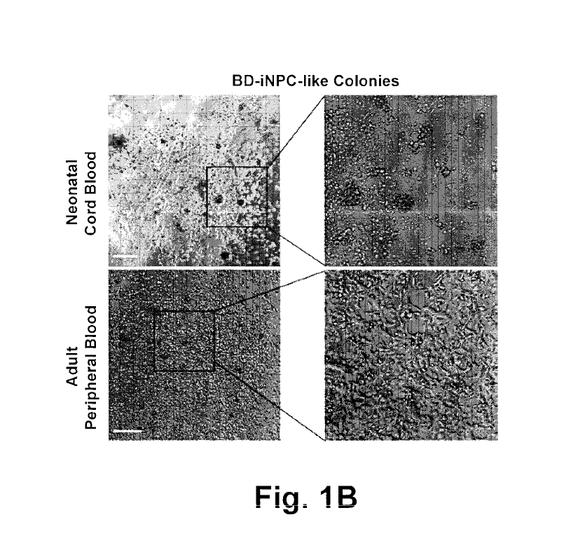

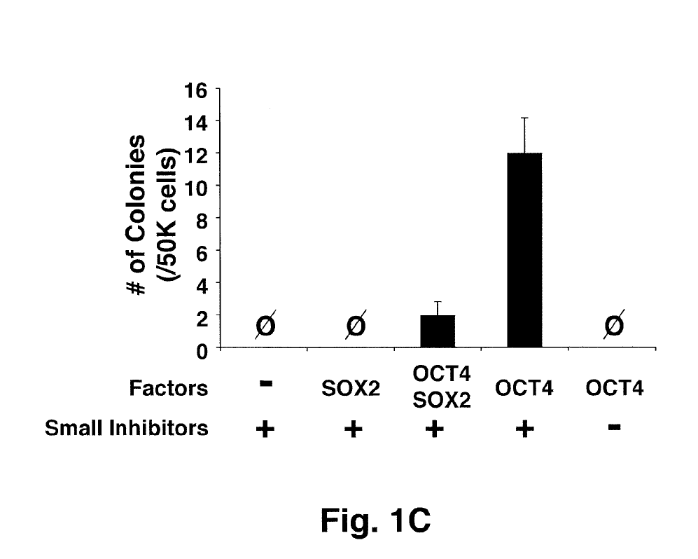

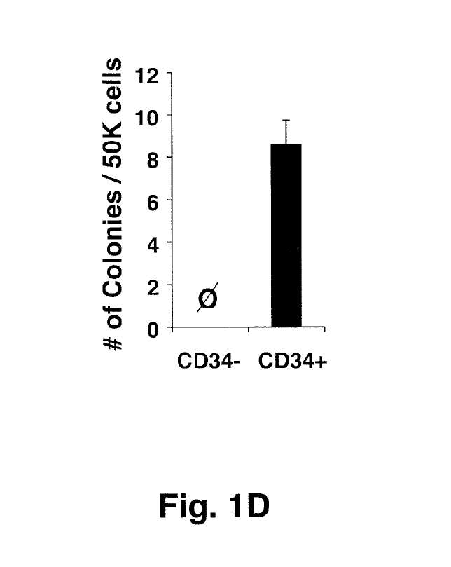

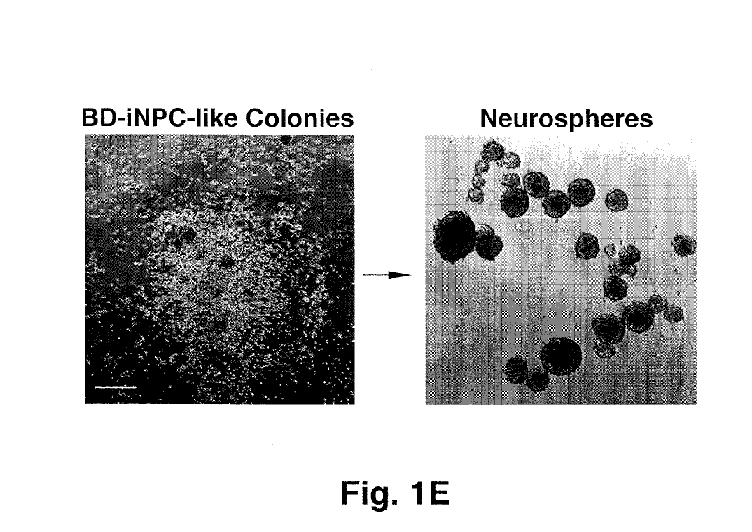

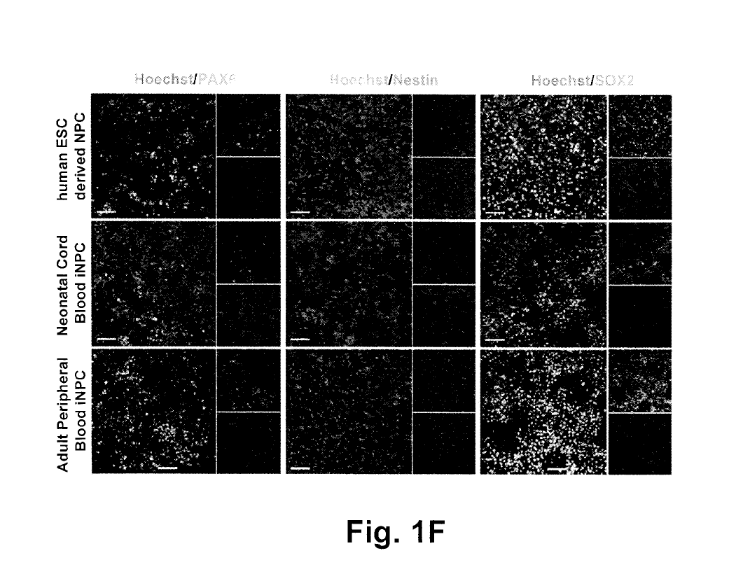

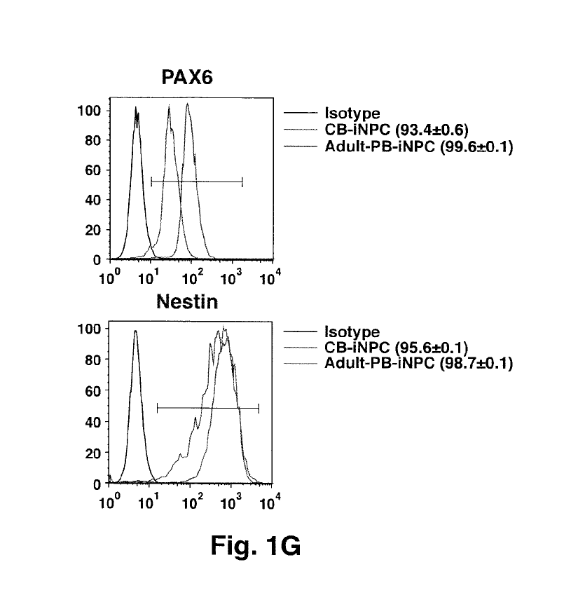

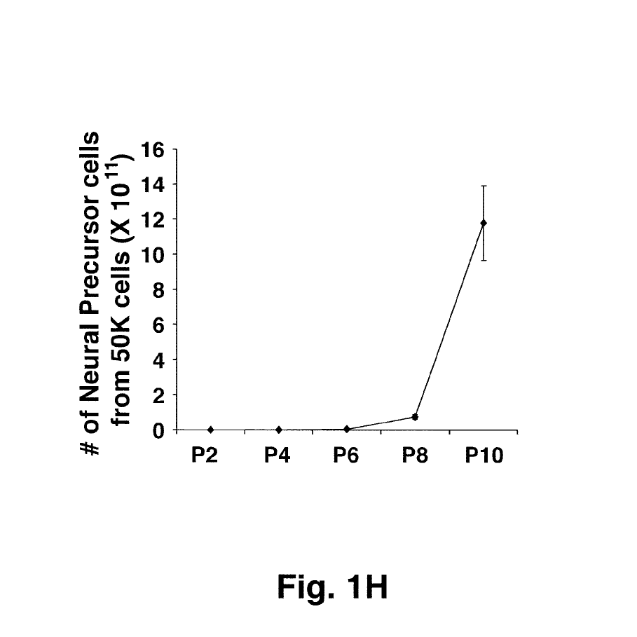

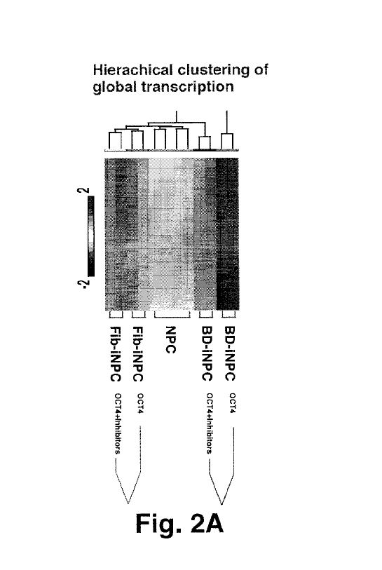

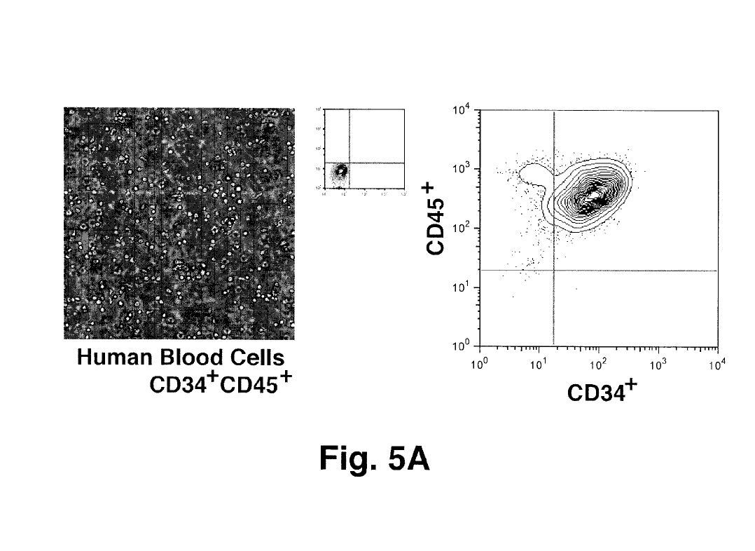

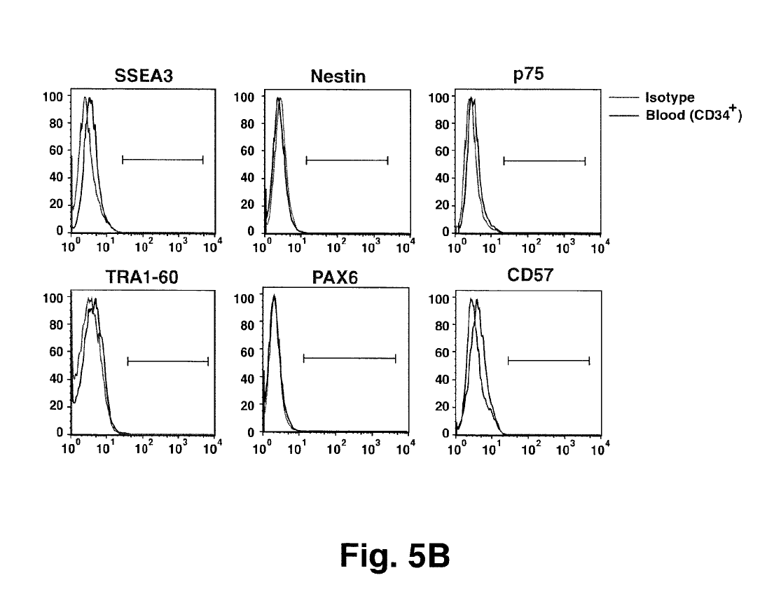

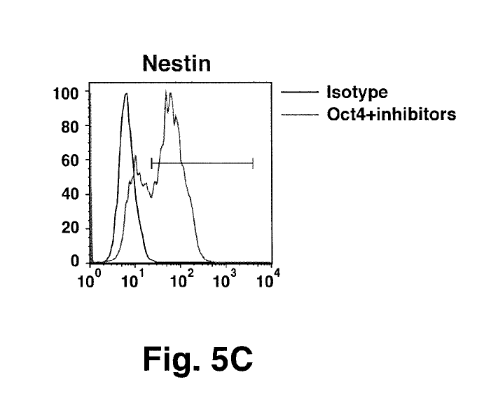

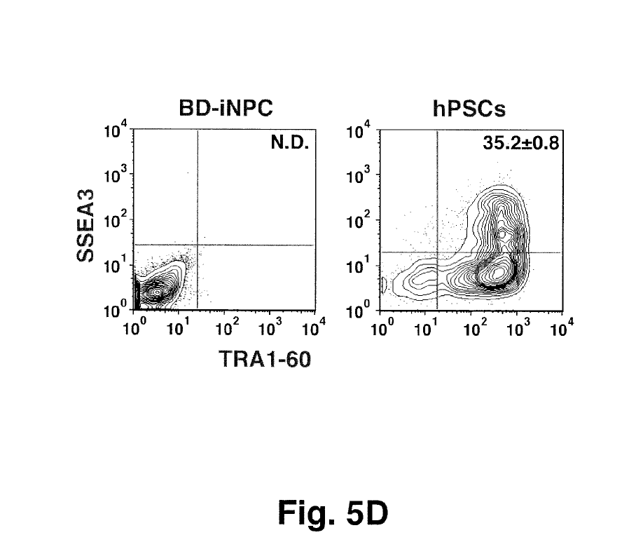

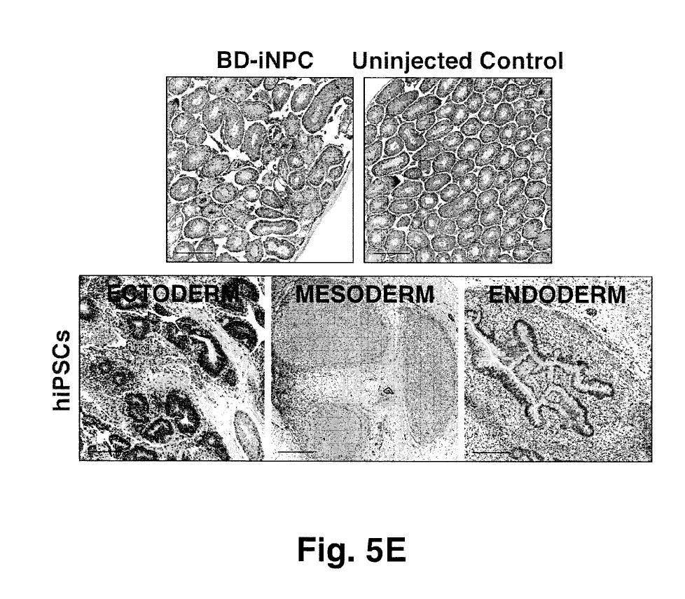

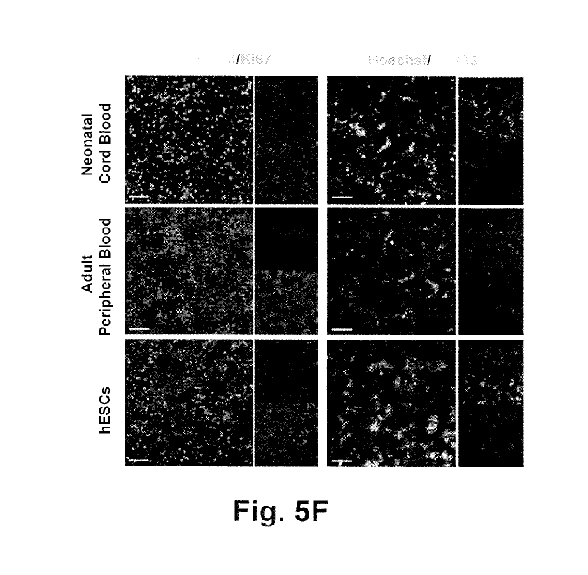

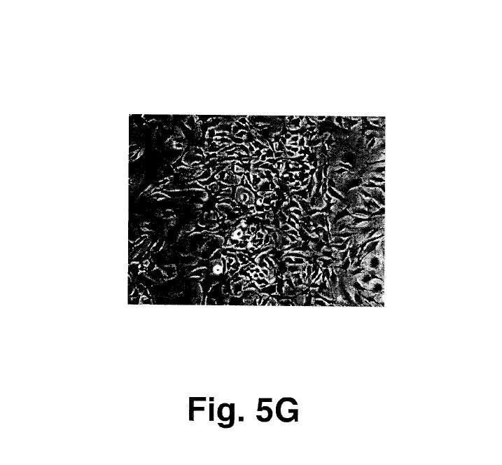

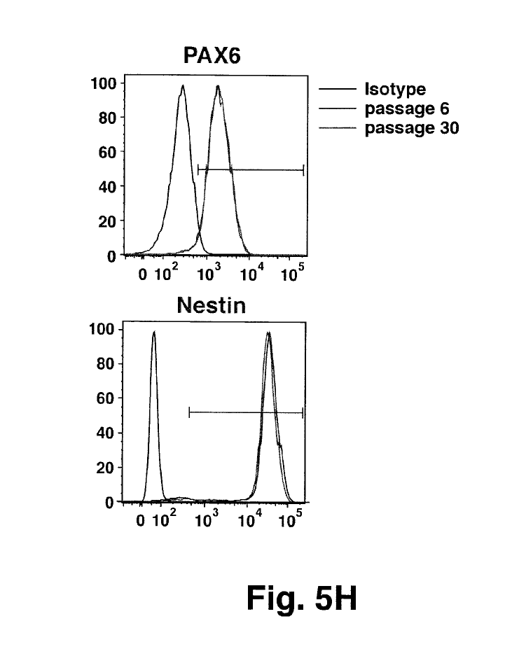

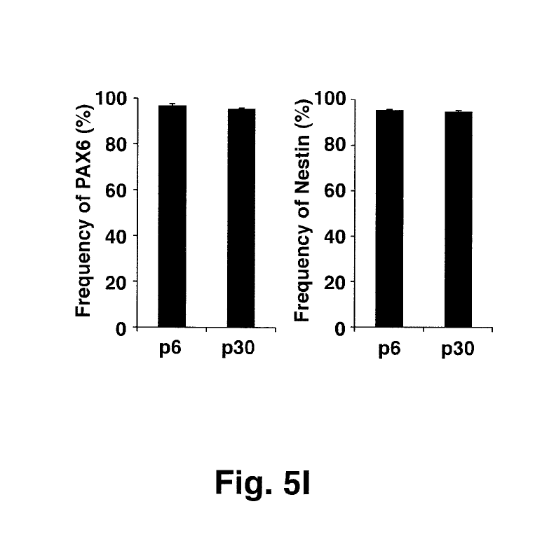

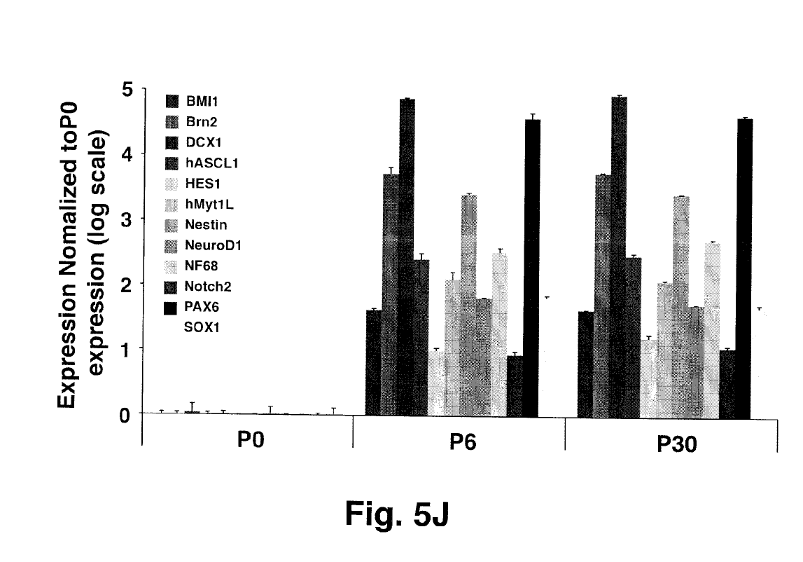

In an effort to make use of readily accessible hematopoietic cells as a starting material to generate neural derivatives, OCT4 based reprogramming (Mitchell et al., 2014a; Mitchell et al., 2014b) to both cord blood and adult peripheral blood progenitors was employed (FIG. 5A). Human blood cells from both sources were negative for pluripotent markers (SSEA3, TRA1-60), early neural markers (Nestin, PAX6) as well as neural crest (NC) markers (p75, CD57) (FIG. 5B), thereby excluding the presence of contaminating cells with pluripotent or NPC features within the starting blood samples. Transduction with OCT4 alone has previously been shown to induce human skin fibroblast conversion to tri-potent neural progenitors (Mitchell et al., 2014a), despite reports suggesting the requirement for a chemically diverse cocktail of inhibitors in addition to OCT4 (Zhu et al., 2014). However, transduction of human blood with OCT4 alone failed to induce production of iNPCs (FIG. 1A-C). As both inhibition of SMAD and glycogen synthase kinase-3β (GSK3β) signaling have been independently reported to efficiently neuralize hPSCs (Chambers et al., 2009), it was examined whether dual inhibition with SMAD and GSK-3 chemical inhibitors could facilitate iNPC generation from blood coupled with OCT4 induced plasticity (FIG. 1A). When human blood progenitors obtained from neonatal cord blood or adult peripheral blood expressing OCT4 were transferred to SMAD+GSK-3 inhibition conditions (SB431542, LDN-193189, Noggin, CHIR99021), iNPC-like clusters appeared within as little as 8-10 days and showed the expression of the neural stem cell marker, Nestin (FIG. 1B and FIG. 5C). Addition of these same molecules to human fibroblasts had no effect on NPC generation (Mitchell et al., 2014a; Mitchell et al., 2014b). As SOX2 has also been implicated in direct-fate reprogramming towards the neural lineage (Ring et al., 2012), the present inventors tested whether SOX2 transduction alone or in combination with OCT4 enhanced iNPC-like cluster formation. No detectable iNPC-like clusters upon expression of SOX2 alone, as well as reduced iNPC formation were found when used in combination with OCT4 (FIG. 1C). The use of OCT4 expression combined with chemical inhibitors was a highly efficient process, and up to 12 putative iNPC-like colonies could be generated from as few as 50,000 (50K) human blood progenitors (FIG. 1C). OCT4-dependent generation of human iNPC colonies could not be established from more mature blood cells devoid of CD34 expression (CD34⁻) and was restricted to the hematopoietic progenitor-containing compartment (FIG. 1D). Furthermore, individual iNPC-like colonies demonstrated robust survival that allowed subsequent collection and re-culturing to promote cell proliferation and expansion into primary neurospheres using suspension culture (Ring et al., 2012) conditions known to support human NPCs (FIG. 1E). The absence of pluripotent markers (TRA1-60 and SSEA3) demonstrated that OCT4 induced iNPCs were not products of intermediate pluripotent states (FIG. 5D), which was further supported by the failure to give rise to teratomas when transplanted into immunodeficient mice (FIG. 5E). The complete absence of a pluripotent cell from human blood derived OCT4-induced iNPC also removes safety concerns regarding potential future use of BD-iNPCs. BD-iNPCs derived from either neonatal cord blood or adult peripheral blood consistently expressed neural stem cell associated markers including PAX6, NESTIN, SOX2 and CD133 similar to control human NPCs (FIG. 1F,G and FIG. 5F,G). Moreover, cultured BD-iNPCs contained ki67 expressing proliferative cells (FIG. 5F) that enabled serial passaging without the loss of NPC marker expression, neural transcriptional programs, or genomic integrity (FIGS. 5H-J and 6A,B). As a testament to their robust practical utility, with an average of as few as 12 iNPC colonies consistently generated from 50K human blood progenitors, as many as 100 million progenitor cells over 10 passages (FIG. 1H) can be generated using this direct conversion approach from human blood samples.

SMAD+GSK-3 Inhibition Facilitates NPC Generation from Human Blood

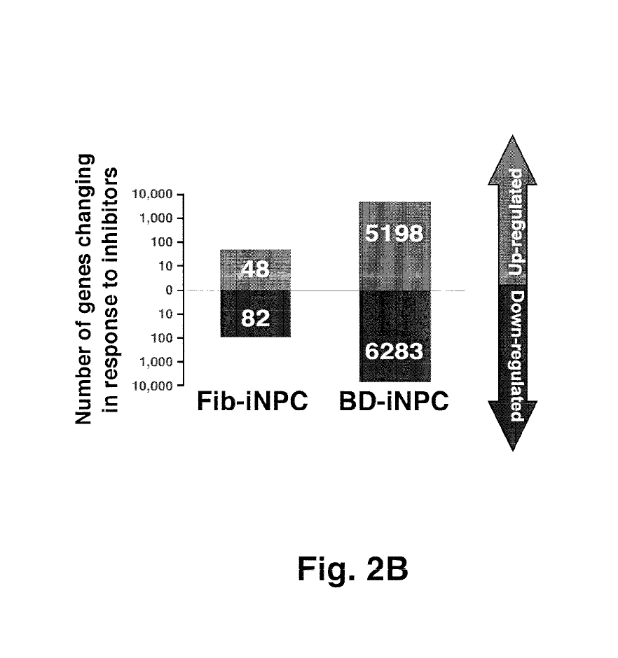

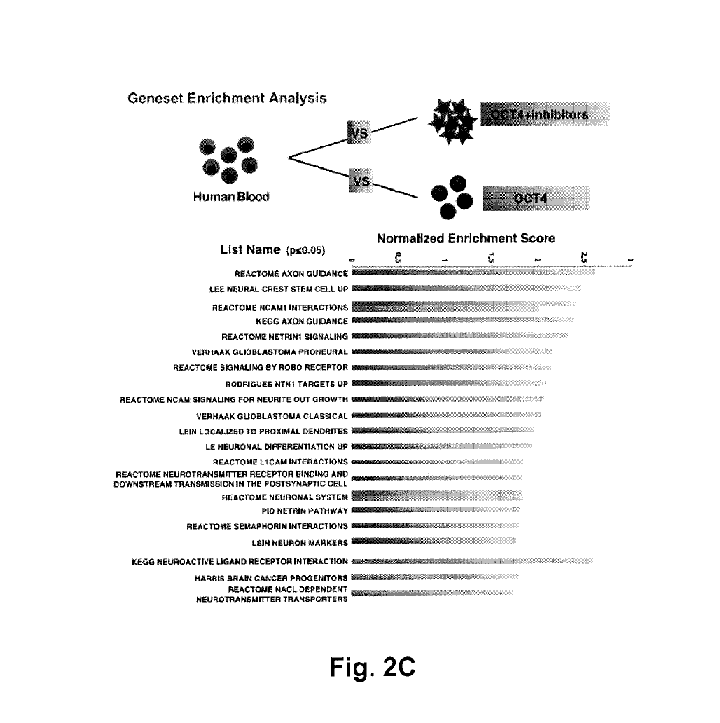

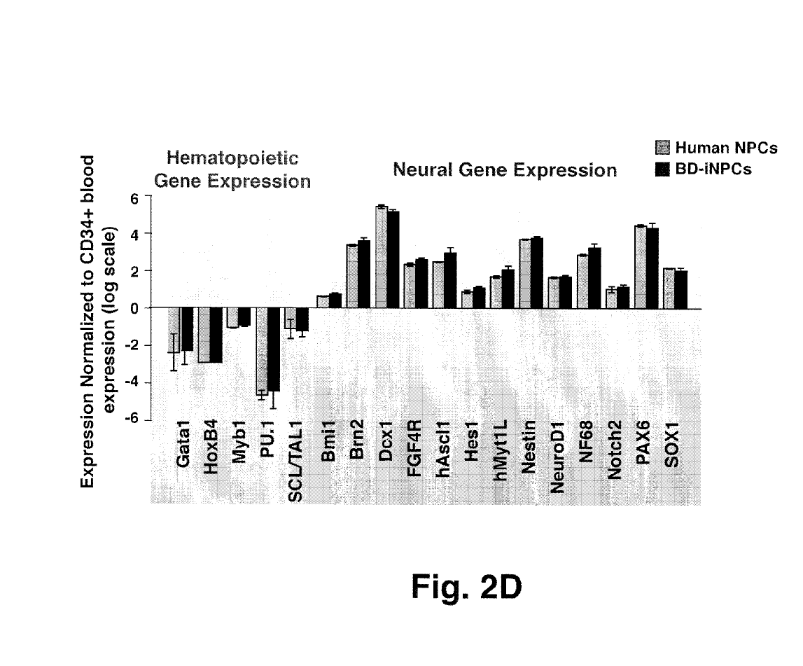

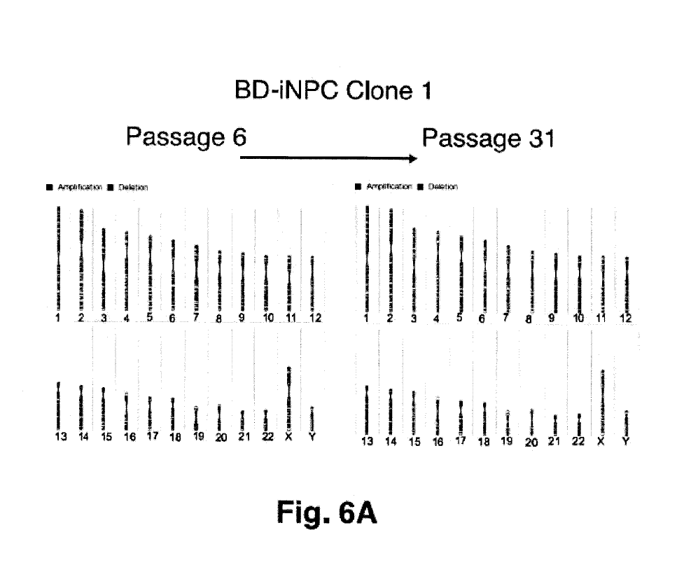

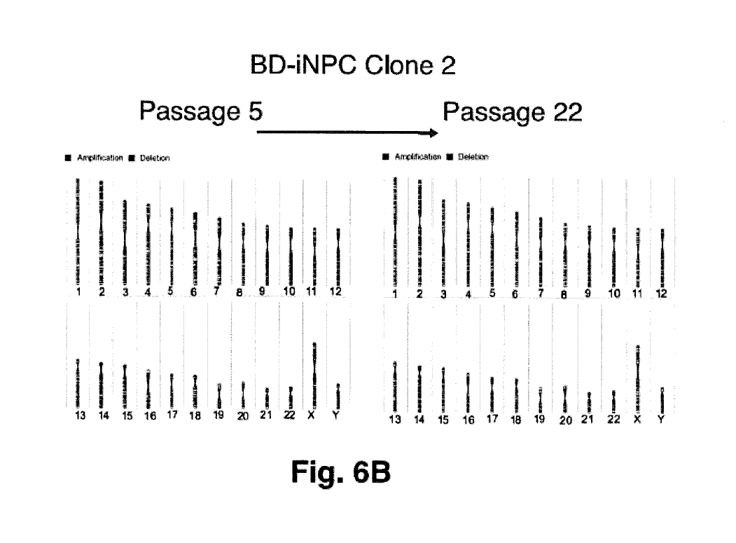

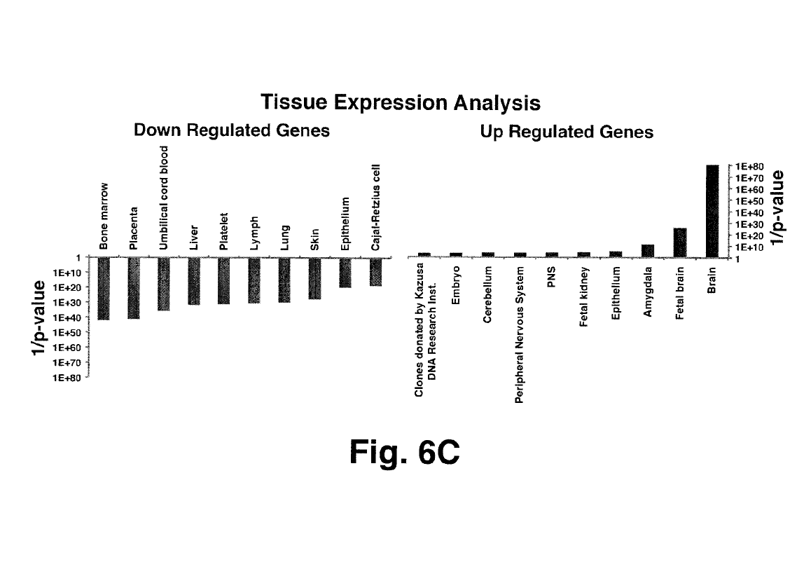

In order to gain a better understanding for the requirement of dual SMAD+GSK-3 inhibition during OCT4 mediated conversion of human blood cells to iNPCs, molecular profiles of blood cells expressing OCT4 were assembled that were either treated or not treated with inhibitors, and were compared to profiles of recently described Fibs-iNPC^{OCT4} that were derived in the same fashion. To evaluate the molecular profiles, hierarchal cluster analysis of global gene expression profiles was performed and SOX2 expressing primary neural stem/progenitor cells isolated from human brain tissue was included as a base of reference (FIG. 2A). As expected, Fib-iNPCs were highly related to primary human NPCs regardless of inhibitor addition (Mitchell et al., 2014b), whereas BD-iNPCs required SMAD+GSK-3 inhibition in order to cluster together with primary NPCs (FIG. 2A). Investigation of differential gene regulation between +/−inhibitor treated fibroblasts and blood cells during generation of NPCs displayed minimal changes in fibroblast transcriptome compared to blood cells, suggesting a unique role for SMAD+GSK-3 inhibition during blood based OCT4 reprogramming (FIG. 2B). In order to classify the gene programs that were specifically regulated in blood cells undergoing OCT4 iNPC reprogramming, gene set enrichment analysis was performed on blood cells+/−inhibitor treatment during derivation of NPCs. The addition of SMAD+GSK-3 inhibition resulted in the enrichment of multiple neural related gene sets that were otherwise not activated in the presence of OCT4 expression alone (FIG. 2C). Furthermore, filtering of both up- and down-regulated genes using the Tissue Expression analysis tool on DAVID Bioinformatics Resource revealed enrichment of down-regulated genes within hematopoietic programs and up-regulated genes within neural programs (FIG. 6). In order to validate the trends from the molecular profiling studies, candidate qPCR was performed on BD-iNPCs for potent hematopoietic and neural progenitor regulatory genes, which confirmed a successful molecular switch from blood to neural progenitors (FIG. 2D). These detailed analyses indicate the processes involved in conversion of human skin fibroblasts to NPCs vs. blood derived NPCs are also molecularly distinct, and reveal a complete conversion of human blood progenitors to NPC fate that is not limited to phenotypic alternations alone.

### BD-iNPCs Expand and Functionally Respond to In Vivo and Directed In Vitro Differentiation Cues

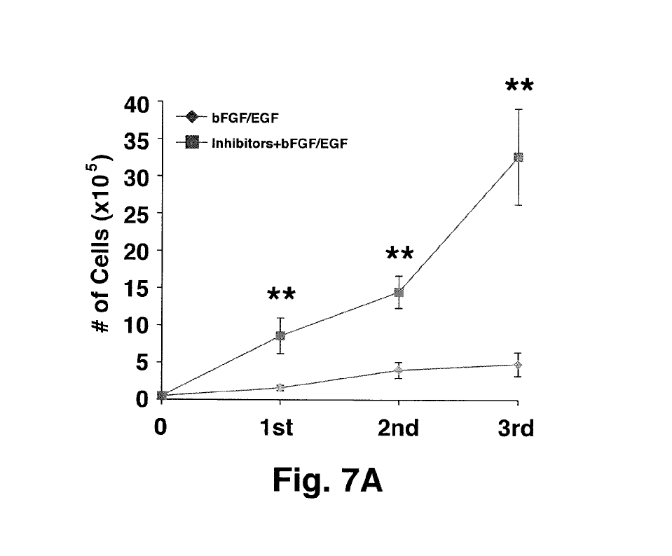

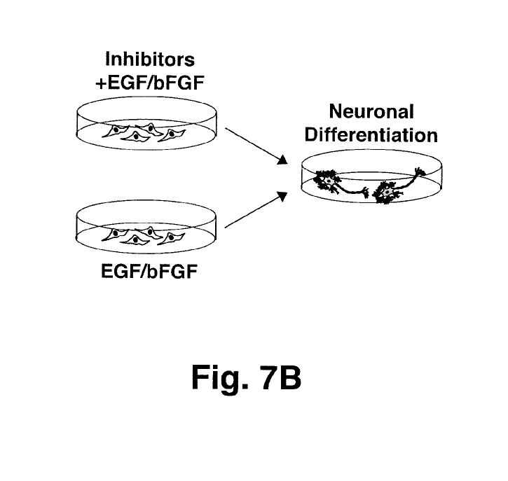

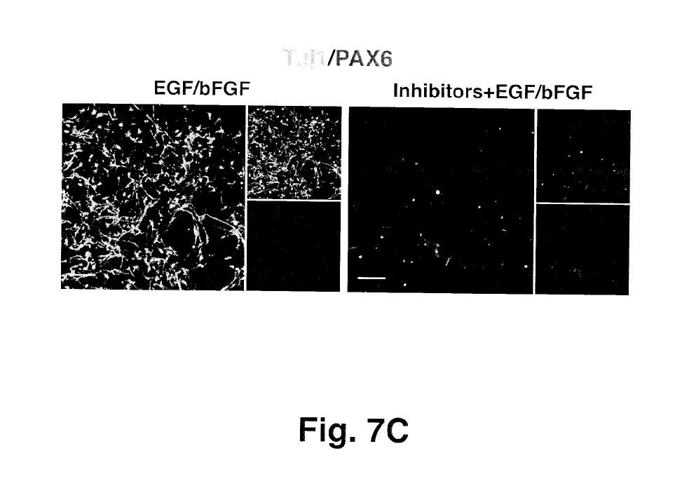

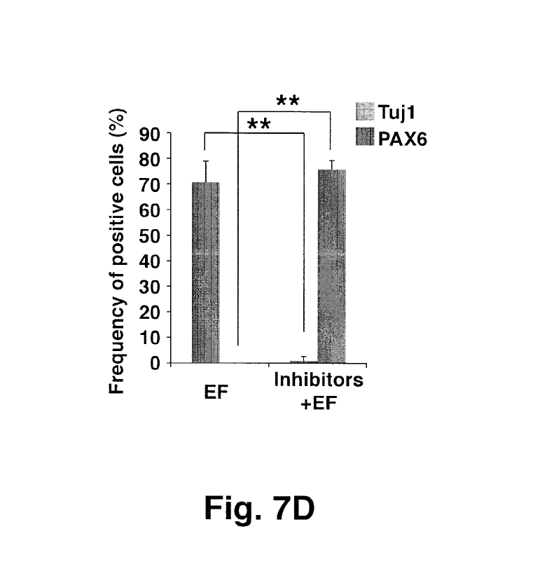

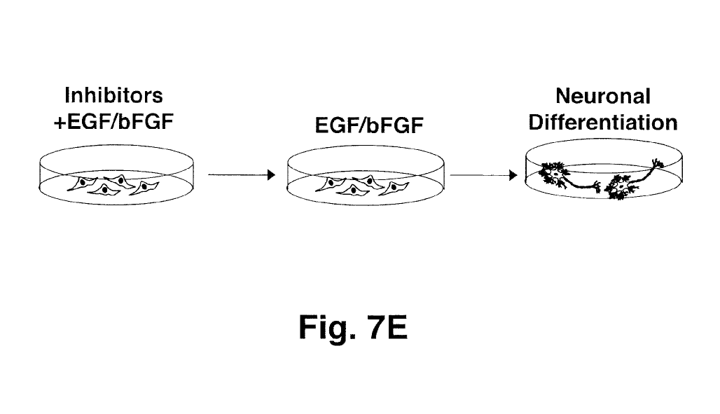

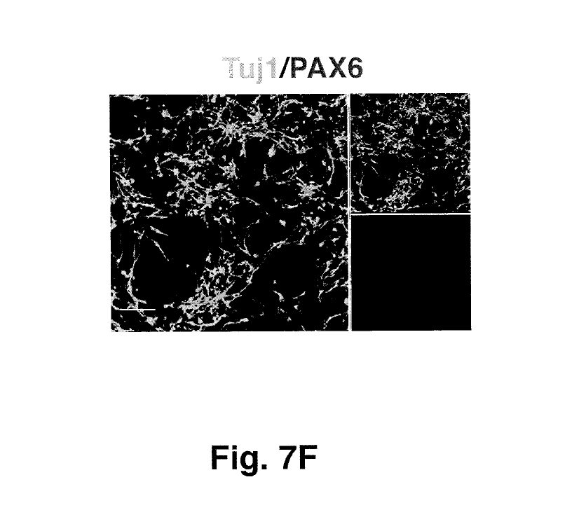

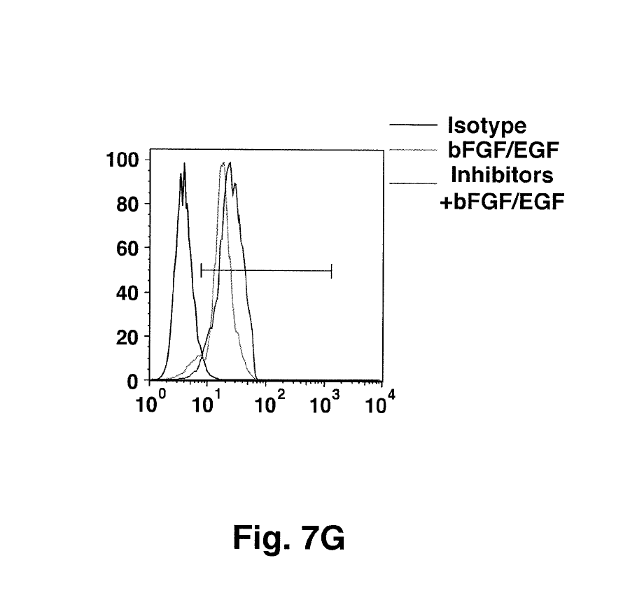

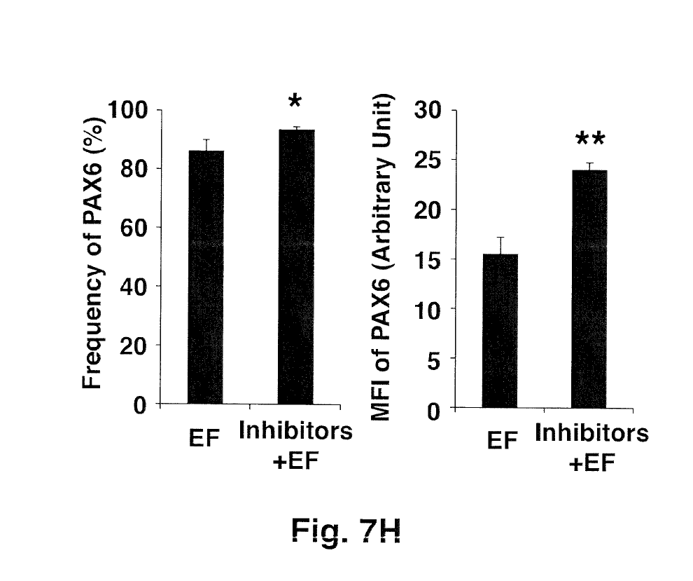

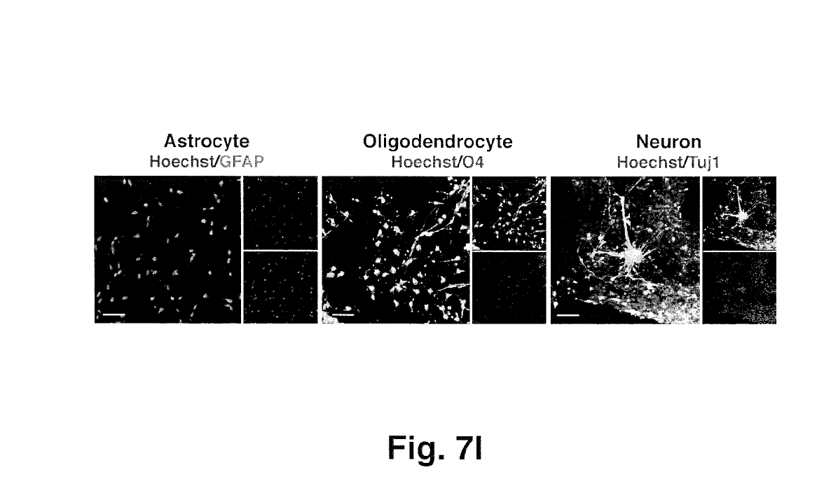

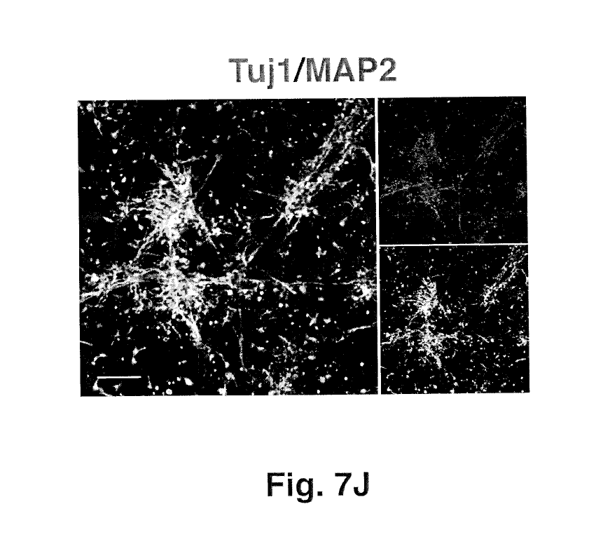

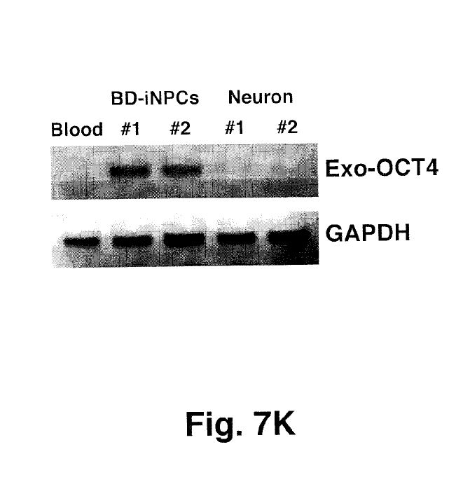

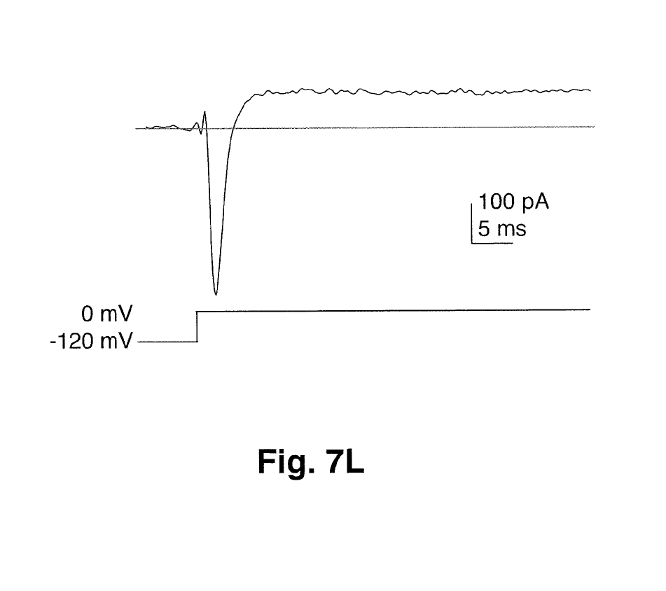

Having established the role for SMAD+GSK-3 inhibition during the initial generation of BD-iNPCs from human blood progenitors, the direct effects on proliferative expansion and developmental potential of the resulting BD-iNPCs were next examined. SMAD+GSK-3 inhibition resulted in enhanced proliferation of BD-iNPCs compared with inhibitor-withdrawn cultures (FIG. 7A). However, enhanced proliferation came at the expense of differentiation, as BD-iNPCs maintained in the presence of SMAD+GSK-3 that were transferred to culture conditions conducive for neuronal-differentiation (FIG. 7B), displayed continued PAX6 expression but failed to upregulate Tuj1 compared to BD-iNPCs where inhibitors were withdrawn (FIG. 7C,D). Despite a clear indication that inhibitor treatment imposed differentiation block, this phenomena was rapidly reversed within one round of passaging in the absence of inhibitors, indicative of successful maintenance of differentiation potential throughout proliferative cycles (FIG. 7E,F). Interestingly, quantified levels of PAX6 in long-term cultures revealed the expression of the NPC marker was higher in the presence of SMAD+GSK-3 inhibitors, although the frequencies of positive cells were comparable (FIG. 7G,H). These results reveal that modulation of SMAD+GSK-3 signaling plays an important role in the regulation of proliferation and differentiation potential of BD-iNPCs.

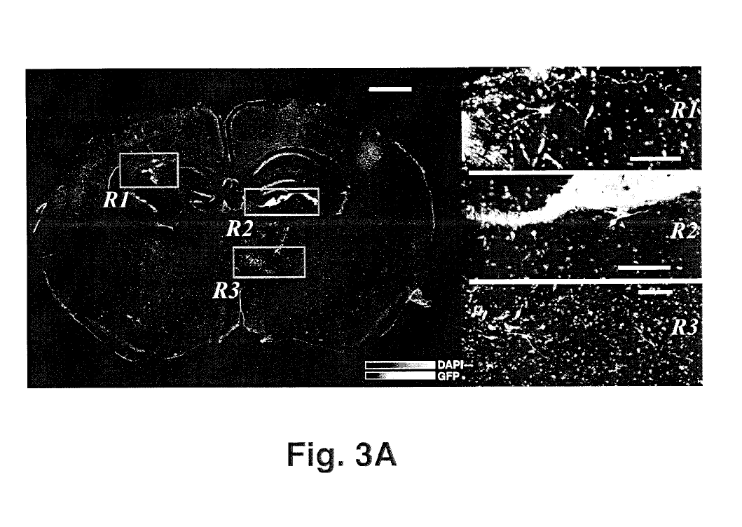

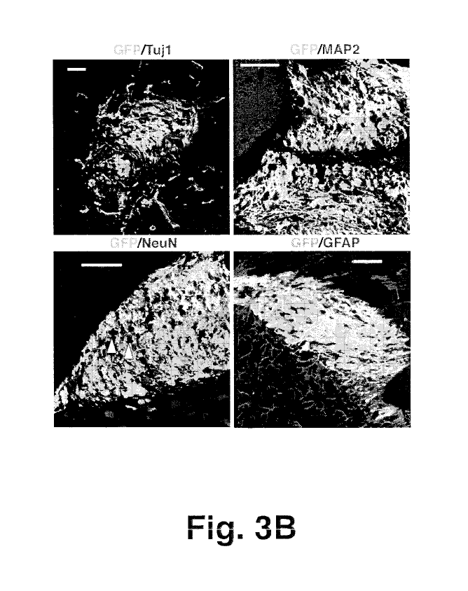

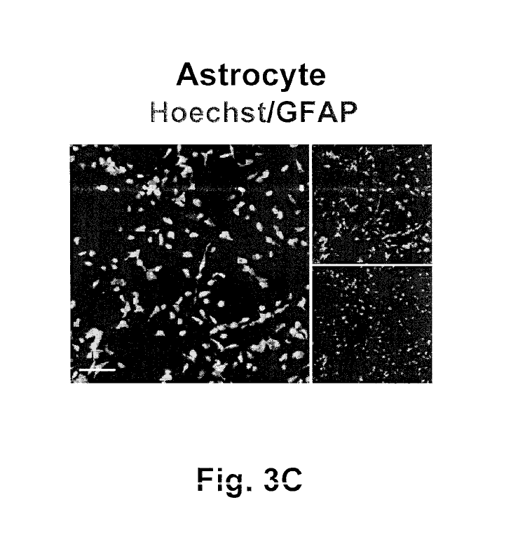

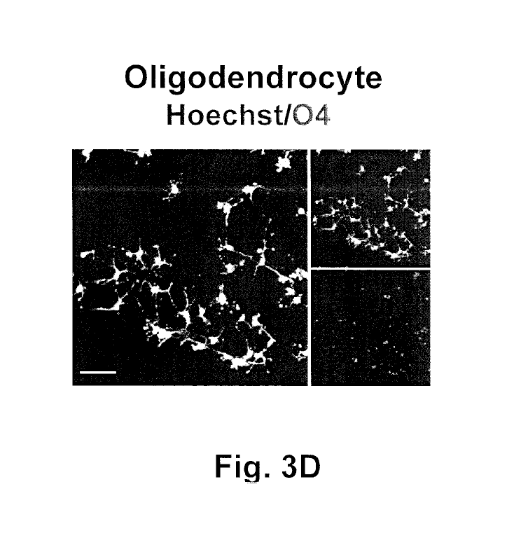

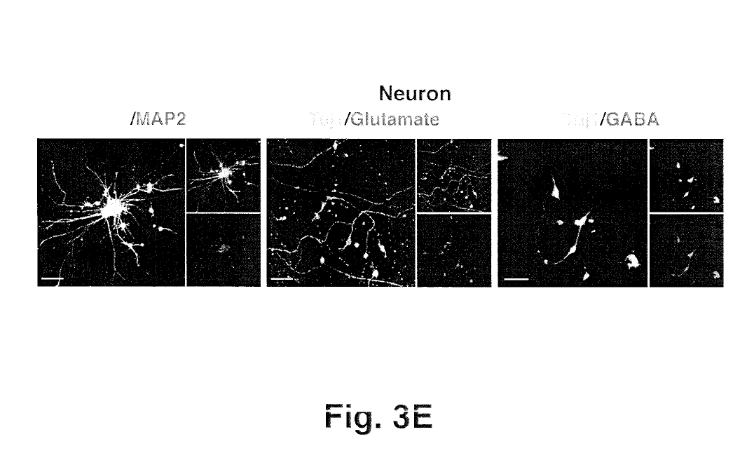

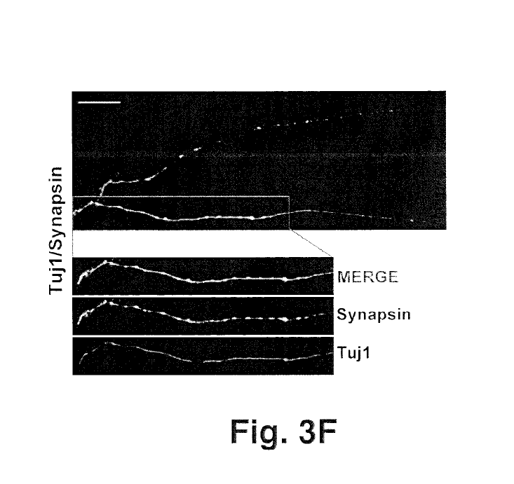

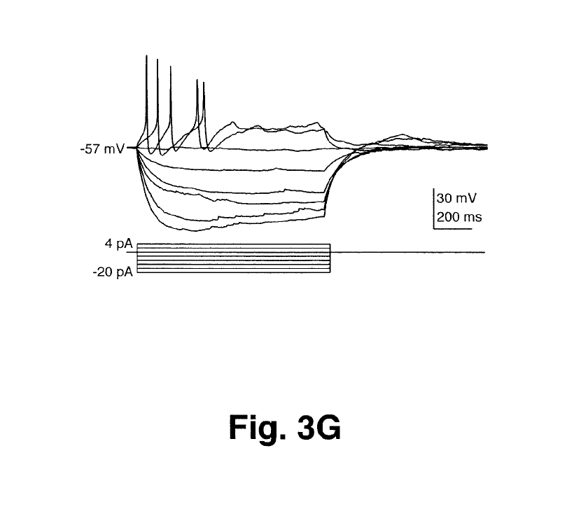

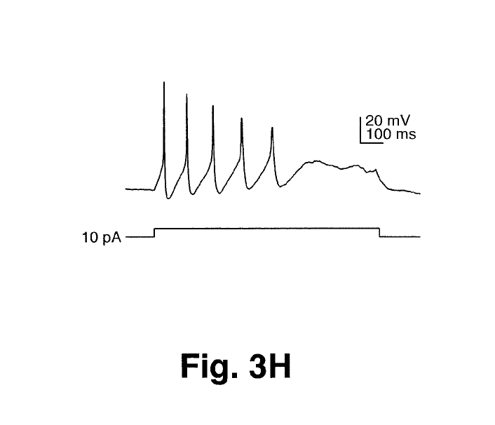

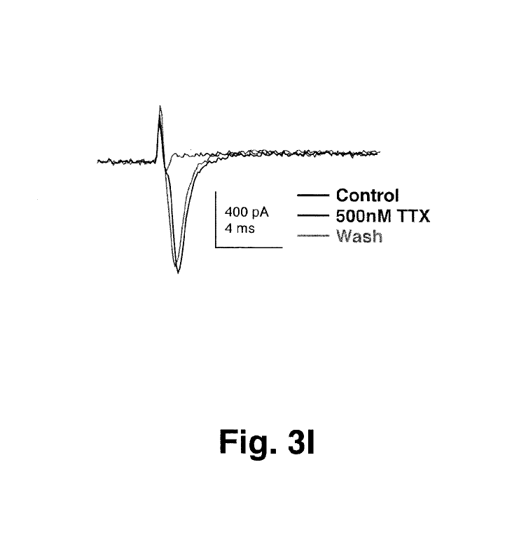

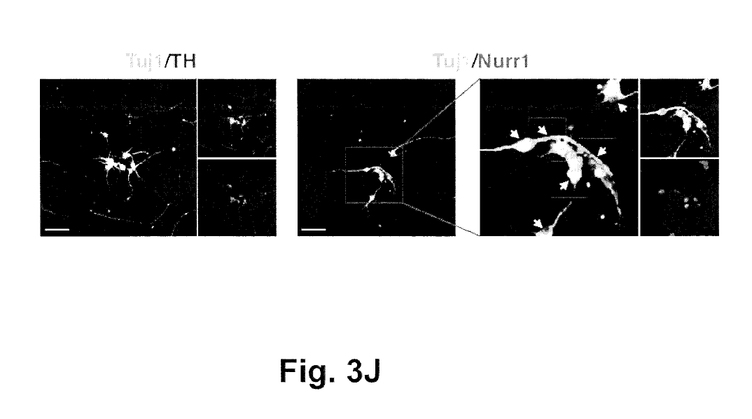

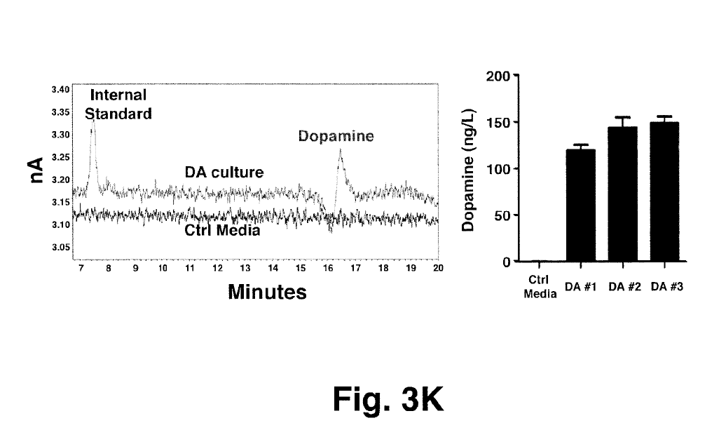

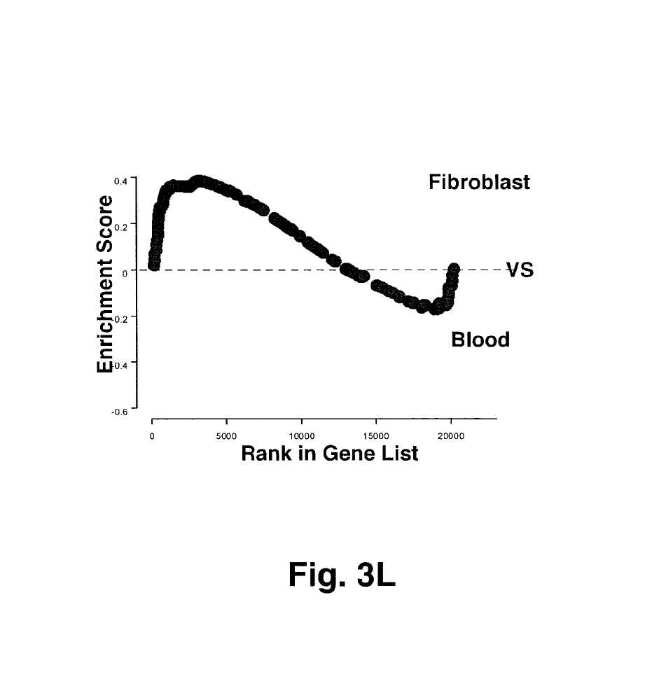

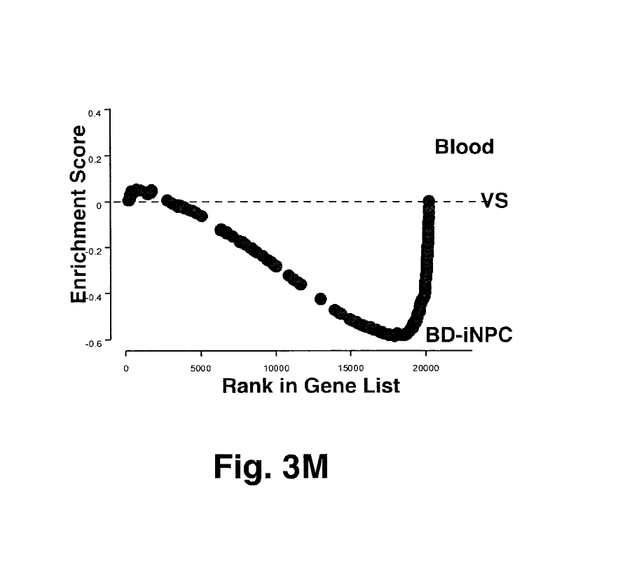

The present inventors next set out to evaluate the developmental potential of OCT4 induced BD-iNPCs by assessing their ability to functionally differentiate in vivo towards the three main neural lineages. BD-iNPCs were transduced with a GFP expressing lentiviral vector and then injected into the brains of p2-p4 mouse pups and allowed to engraft for 3 weeks (Zhu et al., 2014). Analysis of GFP signal from sectioned brain tissue as a surrogate of human engraftment revealed multiple sites containing intact human cells (FIG. 3A). Investigation for differentiated BD-iNPC progeny revealed populations of GFP positive cells that co-expressed both TUJ1 and MAP2 with clear neuronal morphology (FIG. 3B). Moreover, GFP positive human cells were identified that also co-expressed glial fibrillary acidic protein (GFAP), consistent with the presence of astrocytes (FIG. 3B). Despite confirming in vivo differentiation potential towards both neurons and astrocytes, no evidence of in vivo differentiation towards oligodendrocytes was find, a finding not unlike that of other human iNPC studies which have relied on murine xenograft assays (Zhu et al., 2014).

Although in vivo xenograft studies are considered to be the gold standard for many assays of human biology that can otherwise not be measured, in vitro differentiation allows for the directed production of specific cell types that will likely be useful in near term personalized medicine applications of drug screening/testing rather than cellular transplantation. Despite limited detection of oligodendrocytes in the in vivo tests, BD-iNPCs possessed astrocyte and oligodendrocyte differentiation potential in vitro as evidenced by GFAP and O4 expression, respectively, with characteristic morphology similar to differentiated cells from human PSCs (FIG. 3C,D and FIG. 7I). Furthermore, culture conditions for the specification for neuronal development resulted in mature neurons expressing canonical markers TUJ1 and MAP2 (FIG. 3E and FIG. 7J), with the majority expressing high levels of glutamate, consistent with excitatory glutamatergic neurons. Importantly, prior to differentiation BD-iNPCs express OCT4 transgene at observable levels, however similar to previous reports (Mitchell et al., 2014b), OCT4 expression is silenced upon complete differentiation towards mature functional cells types (FIG. 7K). Using specific conditions for GABAergic neurons, GABA-positive inhibitory neurons were successfully generated (FIG. 3E), suggesting BD-iNPCs harbored broad neuronal developmental potential. Moreover, BD-iNPC derived neurons also exhibited a punctate pattern of synapsin expression suggesting the development of synapses (FIG. 3F), which was confirmed using electrophysiological analysis (FIG. 3G-1). Specifically, upon positive current injection, spontaneous repetitive action potential firing was induced (FIG. 3G) and voltage-dependent transient Na⁺ and sustained K⁺ currents were detected (FIG. 7L). Application of tetrodotoxin (TTX) blocked rapidly activating and inactivating inward currents, further demonstrating that the differentiated neurons expressed voltage-activated sodium channels associated with primary neurons (FIG. 3I). Thus, neurons derived from iNPCs appear to exhibit the functional membrane properties and activities of mature neurons. Having observed robust functional neuronal differentiation activity, it was investigated whether BD-iNPCs neuronal differentiation capacity could be expanded into more specialized neurons, such as dopaminergic (DA) neurons, in response to specific instructions. Treatment with Sonic Hedgehog (SHH) and FGF8b (Li et al., 2011) further differentiated BD-iNPCs into neurons expressing tyrosine hydrolase (TH), the rate-limiting enzyme in the synthesis of DA (FIG. 3J). These neurons also expressed the nuclear receptor NURR1 (a.k.a. NR4A2), a key regulator of the dopaminergic system (FIG. 3J). Moreover, the detection of secreted DA in culture medium further supported the presence of functional dopaminergic neurons in vitro (FIG. 3K).

Taken together these results confirm that BD-iNPCs are capable of robust expansion without sacrificing their broad developmental potential, and thereby exhibit the most critical features of bonafide human neural progenitors.

BD-iNPC Generate Functional Nociceptors that Model Chemotherapy Induced Neuropathy

Based on the broad neuronal developmental potential of BD-iNPCs, the transcriptome of BD-iNPCs was further analyzed. These analyses revealed an enrichment of neural crest cell related gene activity compared to that found in blood progenitors (FIG. 2C). Recent work has demonstrated the conversion of human fibroblasts to both putative neural crest (Kim et al., 2014), as well as sensory neurons (neural crest derived peripheral neurons) using typical lineage specifying transcription factor reprogramming strategies (Blanchard et al., 2015; Wainger et al., 2015). Despite a lack of neural crest or sensory neuron functional activity in starting populations of fibroblasts, these human fibroblasts were found to be enriched for neural crest related genes compared to that of human blood cells, suggesting a transcriptionally primed state of neural potential for conversion towards the neural lineage within skin fibroblast cultures not seen in blood progenitors (FIG. 3L). Therefore, in contrast to skin fibroblasts, BD-iNPC conversion involves de novo acquisition of neural crest related gene expression (FIG. 3M). Based on this observation, it was hypothesized that their developmental potential may extend to the peripheral nervous system derivatives, such as sensory neurons.

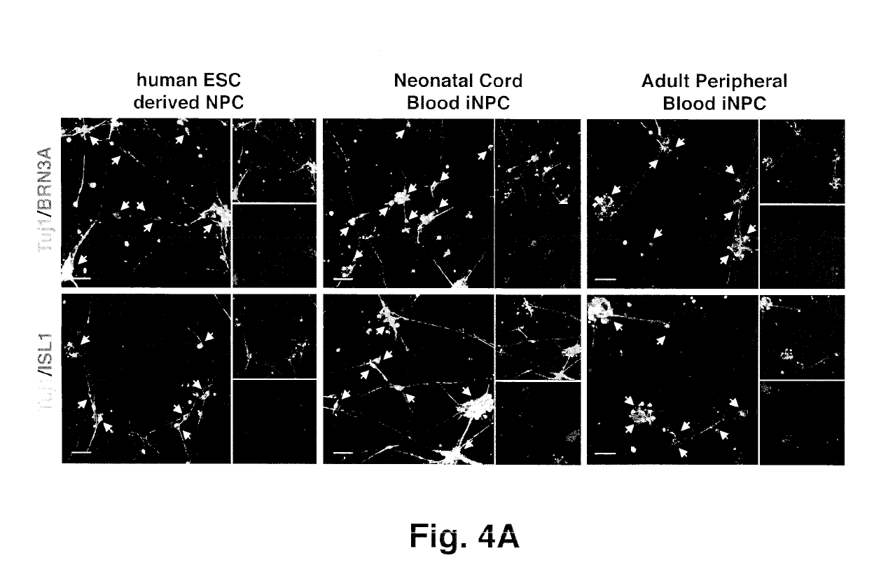

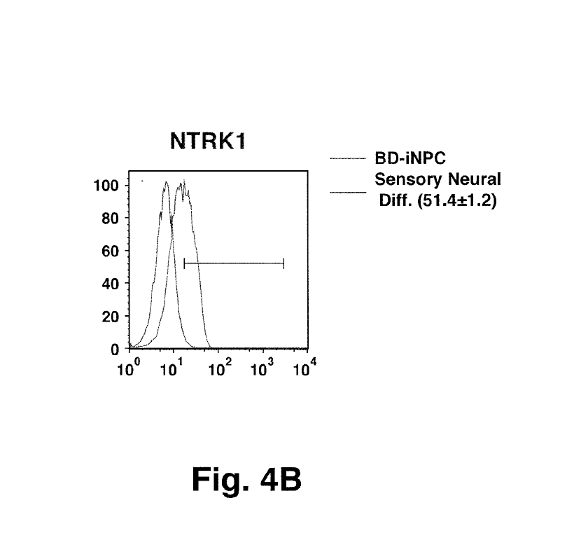

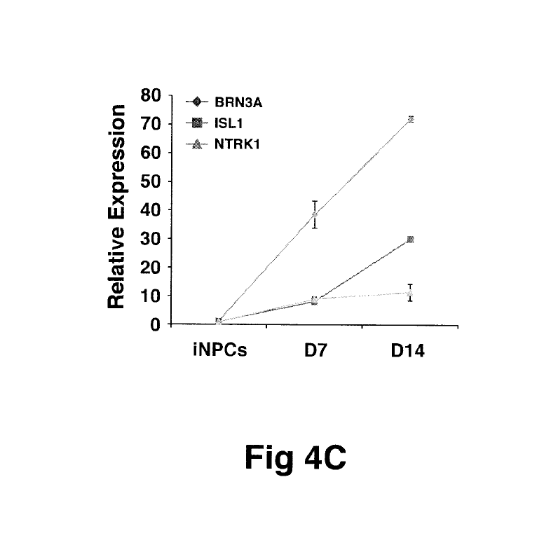

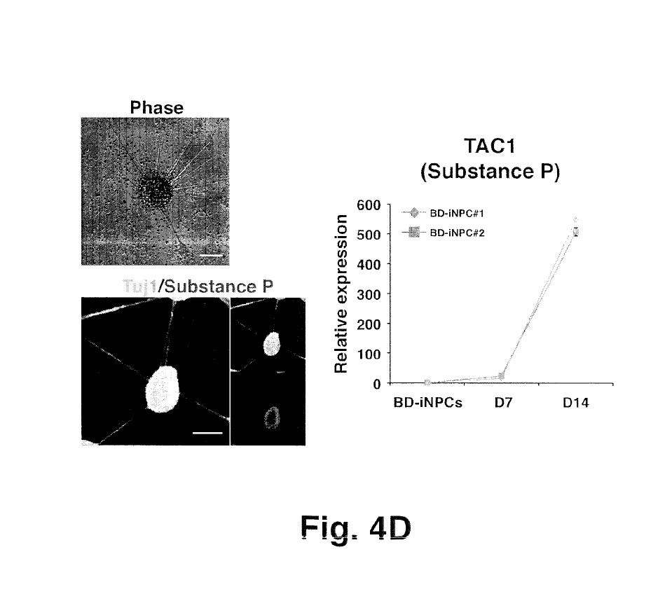

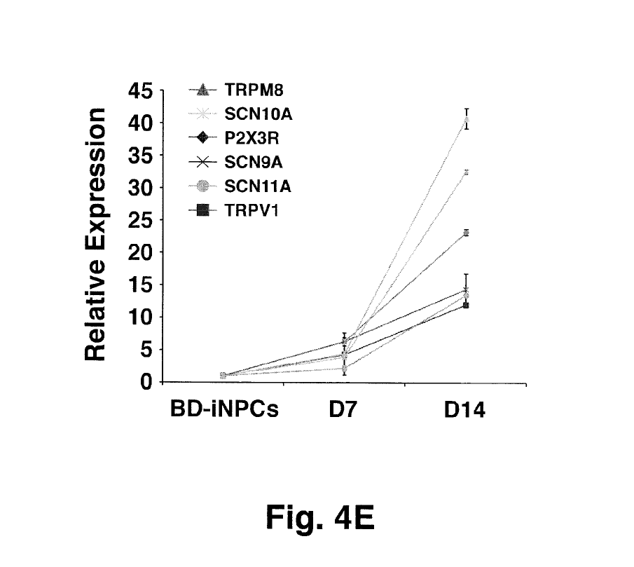

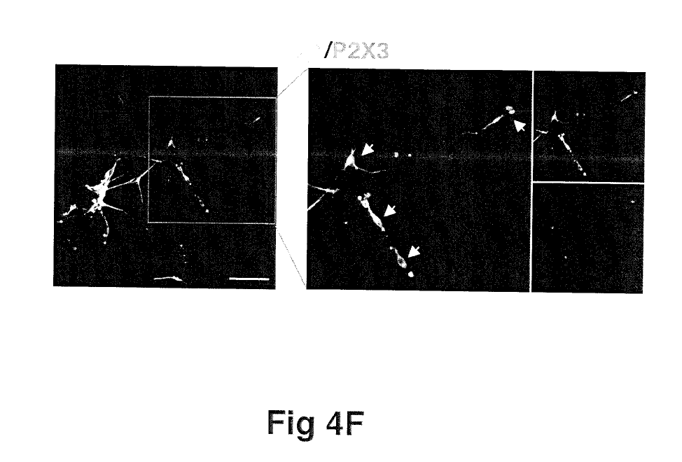

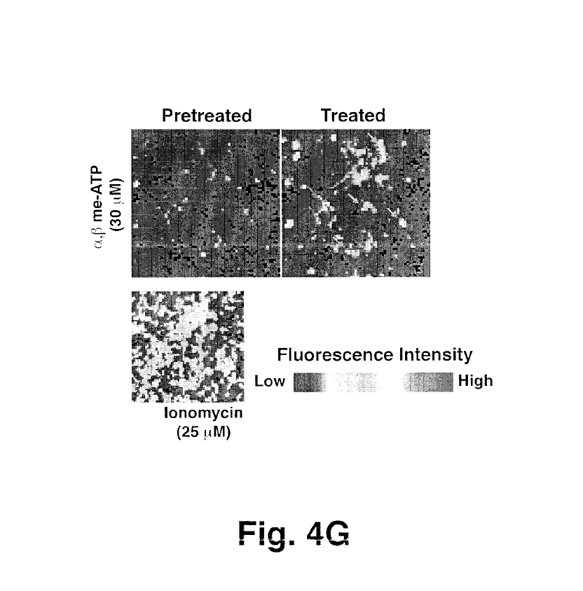

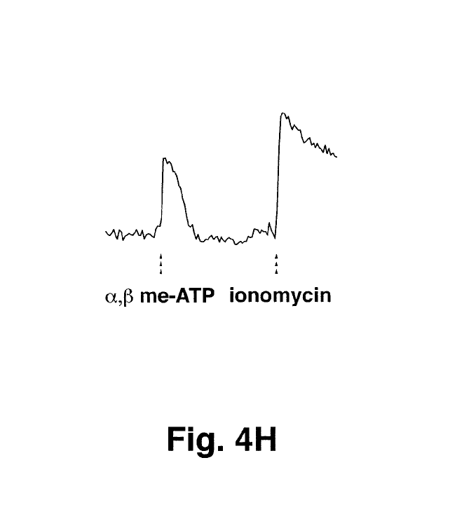

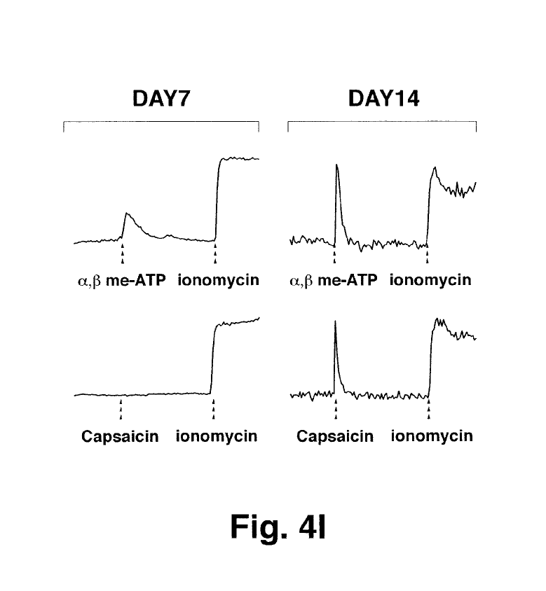

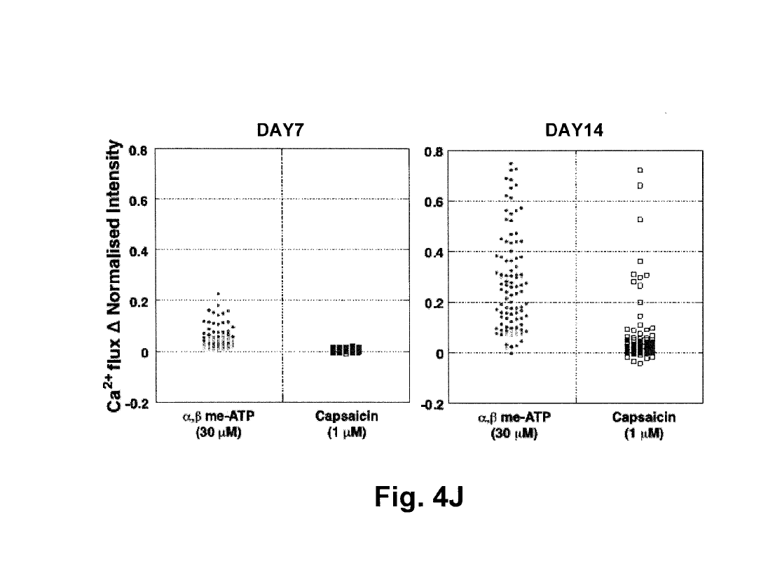

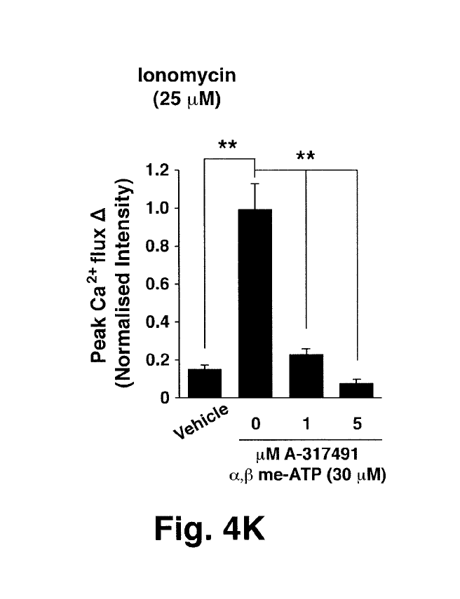

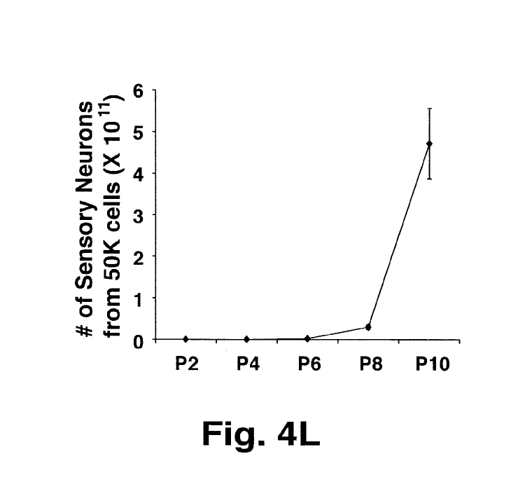

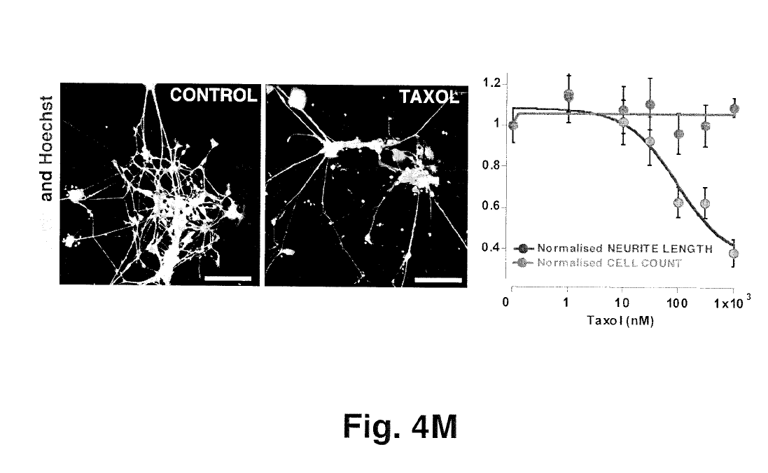

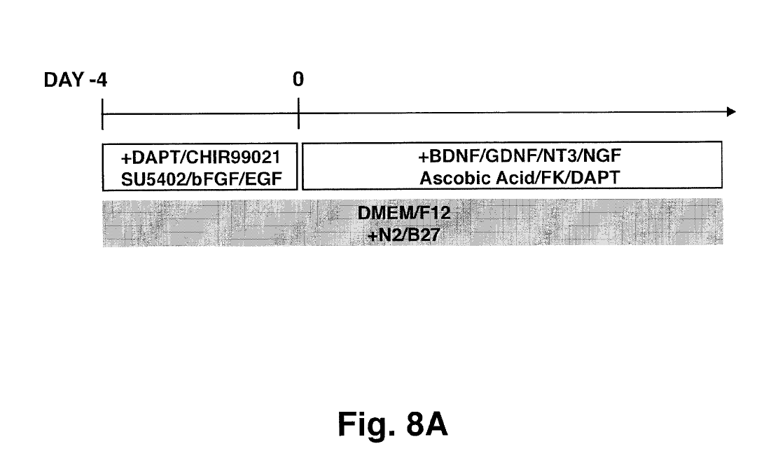

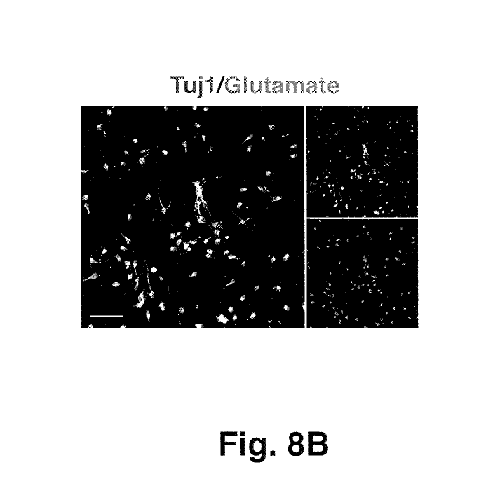

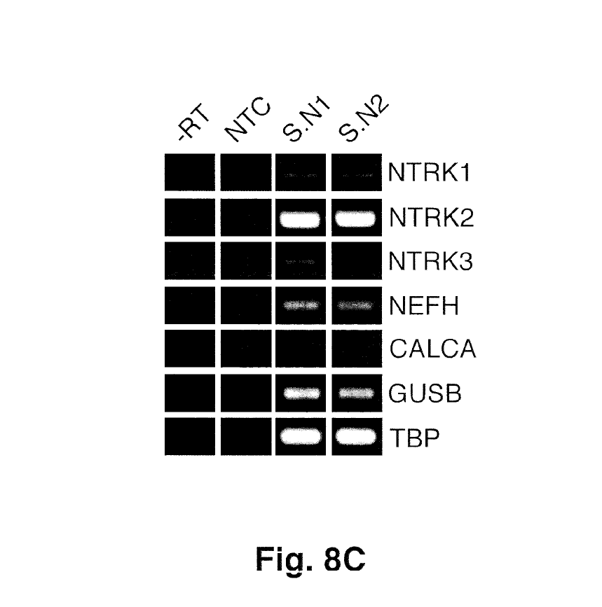

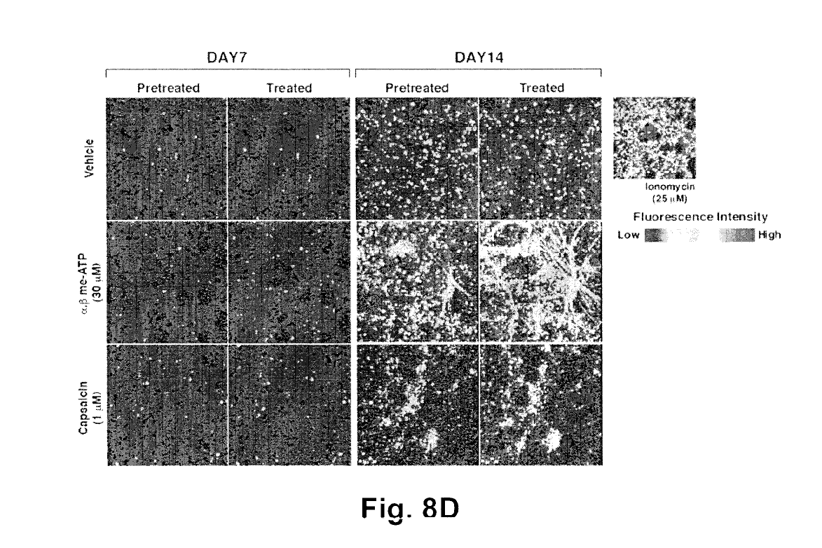

Recent work with pluripotent cells has demonstrated that combined small-molecule inhibition (SU5402, DAPT and CHIR99021) converts human pluripotent cells into sensory neurons (nociceptors) (Chambers et al., 2012). Based on previous reports (Chambers et al., 2012; Guo et al., 2013), modifications to this procedure were made (FIG. 8A) and whether these small-molecule inhibitors could induce generation of nociceptive sensory neurons from directly converted human neonatal and adult BD-iNPCs was tested. The canonical sensory neuronal markers ISL1 and BRN3A were expressed within differentiated neuron preparations from cord blood and adult peripheral blood BD-iNPCs, in a similar fashion as hESC-derived cells shown previously (FIG. 4A). In addition, sensory culture derived neurons expressed glutamate, consistent with an excitatory glutamatergic neuronal phenotype (FIG. 8B) and demonstrated transcript level expression of sensory neuron related genes such as NTRK1, 2, and 3 receptors, neurofilamin heavy chain peptide (NEFH) and calcitonin related peptide alpha (CALCA) (FIG. 8C). These results confirmed that upon treatment with appropriate culture conditions, BD-iNPCs were capable of generating putative sensory neurons; supportive of the theory that BD-iNPC developmental potential extends to PNS related progeny.

Given strong clinical interest for furthering understanding of neurological pain and neuropathy conditions (Bennett and Woods, 2014; Pino, 2010a), combined with the notion that nociceptive neurons (NTRK1 expressing) can be functionally assayed (Blanchard et al., 2015; Wainger et al., 2015), the efforts were focused on nociceptive (NTRK1) neuron generation from BD-iNPCs for further characterization and optimization for use. Analysis of NTRK1 expression at the protein level, revealed approximately 50% of differentiated cell positivity of putative nociceptors (FIG. 4B). Over 14 days, analysis of ISL1, BRN3A and NTRK1 expression indicated that putative nociceptive sensory neurons could be sustained and continually generated from differentiating BD-iNPCs over time (FIG. 4C). Induced neurons were often organized into ganglia-like structures in long-term culture and expressed Substance P (TAC1) indicating the presence of peptidergic nociceptors (FIG. 4D). Moreover, the expression of nociceptor-specific channels and receptors were upregulated during sensory neural induction (FIG. 4E). Expression of the purinergic receptor, P2RX3, considered a unique phenotype of human sensory neurons (Jarvis et al., 2002), was confirmed by immunofluorescence analyses (FIG. 4F).

Functionally, human cord blood and adult BD-iNPC differentiated neurons were evaluated using calcium flux in response to α,β-methylene-ATP, a selective agonist of P2X₃ (FIG. 4G,H) (Jarvis et al., 2002). Although neurons at day 7 post-induction showed expression of putative sensory neuron markers (FIG. 4C), only a minimal response to α,β-methylene-ATP was detectable, whereas continued culture to day 14 allowed a robust response to manifest (FIGS. 41,J and 8D). This indicates that the development of phenotype alone does not suggest functional capacity in terms of ligand responsiveness—an important caution in pragmatic use of converted human neuronal cell types. Furthermore, both the TRPV1 vanilloid receptor agonist capsaicin and P2X₃ agonist alpha, beta-methylene ATP (α,β-meATP) could evoke calcium transients in BD-iNPC derived neurons (Caterina et al., 1997), demonstrating functional activity of nociceptive sensory neurons (FIGS. 41,J and 8D). Importantly, A-317491, a selective P2X₃ inhibitor, significantly decreased this response (FIG. 4K), providing evidence that the α,β-methylene-ATP mode of action was indeed through activation of P2X₃ receptors (FIG. 4K). These findings demonstrate the differentiation potential of BD-iNPCs into nociceptive sensory neurons, which, when combined with their expansion capacity, raises the possibility of generating sufficient quantities of these specialized cells for drug discovery, toxicity and screening applications. It was estimated that a single round of reprogramming could support the generation of as many as 100 million sensory neurons (FIG. 4L) from 50K human blood cells.

Approximately 30 to 40 percent of cancer patients experience the cancer treatment complication of chemotherapy-induced peripheral neuropathy (CIPN) (Pino, 2010b) through the direct impact of the drug on nerve fibers causing nerve degeneration and axon dieback (Boyette-Davis et al., 2011). whether BD-iNPC derived sensory neurons showed similar response to chemotherapy treatment in vitro was tested. Forty-eight hours after treatment with Taxol, neurites of sensory neurons generated from human blood were quantified and showed a dose-dependent reduction in length without concomitant loss of viability (FIG. 4M). This miniaturized and automated approach illustrates the potential utility of these converted cells as an in vitro model of human axonopathy for drug-discovery and form the basis to expand to other PNS and CNS disorders.

### Discussion

The present inventors provide evidence that small molecule inhibitors targeting SMAD+GSK3 enable ectopic expression of OCT4 to directly convert human blood progenitors into proliferative, non-tumorigenic neural precursors with unique multipotent developmental properties that includes generation of both dopaminergic and sensory neurons. Unlike skin fibroblasts with hallmarks of neural lineages, purified CD34⁺CD45⁺ blood is devoid of ectoderm derived cells, and as such BD-iNPCs represent evidence for epigenetic conversion of cell fate state from one developmentally distinct cell type to another (Rieske et al., 2005). Within the context of fibroblast reprogramming, expression of OCT4 and the addition of basal neural progenitor culture conditions is sufficient to support conversion towards iNPCs (Mitchell et al., 2014b) whereas generation of BD-iNPCs shown here is highly dependent on the usage of SMAD+GSK-3 inhibition. Previous attempts to convert human hematopoietic tissue towards the neural lineage were restricted to the use of neonatal cord blood derived MSCs (Yu et al., 2015) or have resulted in the production of neuronal restricted progenitors with limited proliferative potential (Castano et al., 2014). The current study defining BD-iNPCs demonstrates tri-lineage neural progenitor cells produced from direct conversion of adult human blood.

The present disclosure provides a practical and simple approach for generating neural progenitor cells capable of nociceptive neuron differentiation. Although recent work using fibroblasts has demonstrated successful conversion towards pain sensing neurons, these studies require a multi-factor trans-differentiation strategy that bypasses the neural progenitor state (Blanchard et al., 2015; Wainger et al., 2015). As such, each resulting cell is unique from one another given the heterogeneity of fibroblast populations and complex multi-vector integration. BD-iNPCs could aid in realizing goals of better understanding the peripheral-neuropathy component of pain associated with complex disorders such as diabetes and chemotherapy, as well as primary pain that often precedes motor-dysfunction in Parkinson's patients by several years (Tesfaye et al., 2013).

### Materials and Methods

Cell Culture and Derivation of iNPCs

To derive iNPCs, purified CD34⁺ cells from cord blood or adult mobilized peripheral blood were transduced with OCT4 lentivirus in the presence of SCF, Flt-3L, IL3, and TPO cytokines (R&D System). After 48 hr, CD34⁺ blood cells were cultured on Matrigel (BD Biosciences) or irradiated MEFs with reprogramming media and bFGF (R&D System) for 5 days. Cells were then switched to basal NPC media (DMEM/F12, 1×N2, 1×B27 (Invitrogen)) supplemented with SB431542 (Stemgent), LDN-193189 (Stemgent), Noggin (R&D System) and CHIR99021 (Stemgent). After 10-14 days neural precursors-like colonies were manually picked, transferred to Polyornithine/Laminin (POL)-coated culture plates for propagation with neural induction medium supplemented with bFGF and EGF (R&D System). Primary neurosphere culture was used to further enrich iNPCs. Experiments using adult peripheral blood derived iNPCs are shown in FIGS. 1B, 1F, 1G, 4A, 4G, 4H, 8J.

iNPC Differentiation

For neuronal differentiation, basal media was supplemented with retinoic acid (Sigma), forskolin (Stemgent), BDNF, GDNF (R&D System) and ascorbic acid (Sigma). For Astrocyte differentiation, media was supplemented with 5% FBS. For oligodendrocyte differentiation, basal media was supplemented with SHH C25II, bFGF and PDGF (R&D System) for 7 days. Afterwards, PDGF and bFGF were replaced by 3,3,5-triiodothyronine (T3) hormone (Sigma), Noggin, IGF1, NT3 and forskolin adapted from (Lujan et al., 2012; Najm et al., 2013).

Generation of Neuronal Subtypes from iNPC

For GABA neuron induction, the present inventors adapted: (Barberi et al., 2003; Ma et al., 2012); iNPCs were cultivated in basal medium supplemented with SHH C25II without EGF. After 7 days, media was supplemented with, VPA, NT4, BDNF, GDNF, IGF1 and forskolin for 21 days. For DA neuron induction, the present inventors adapted: (Kriks et al., 2011; Li et al., 2011), iNPCs were cultured in basal medium supplemented with SHH C25II and FGF8 (R&D System) without bFGF/EGF. After 7 days, media was supplemented with, BDNF, GDNF, TGFβ3, ascorbic acid, forskolin and DAPT (Sigma) for 21 days. For nociceptive sensory neurons, the present inventors adapted: (Chambers et al., 2012; Guo et al., 2013; Lee et al., 2012). Briefly, iNPCs were cultured in basal medium supplemented with SU5402, DAPT and CHIR99021. After 4 days, media was supplemented with, BDNF, GDNF, NGF, NT3 (R&D System), ascorbic acid and forskolin for 7-14 days until the desired maturation stage for a given experiment.

### Teratoma Assay

iNPCs or undifferentiated hPSCs (1×10⁶ cells/mouse) were IT injected into NOD/SCID mice as described previously (Werbowetski-Ogilvie et al., 2009). 8 weeks post-injection, mouse testicles were harvested, sectioned and stained with hematoxylin and eosin. Images were acquired using ScanScope CS digital slide scanner (Aperio, Calif., USA).

### Flow Cytometry

Cells were fixed using the BD Cytofix/Cytoperm kit (BD bioscience), including 4% (vol/vol) paraformaldehyde fixation step. Fixed cells were stained using the following antibodies: SSEA3, TRA1-60, PAX6, p75, CD57 (BD Biosciences), Nestin, NTRK1 (R&D Systems). Unconjugated antibodies were visualized with appropriated fluorochrome conjugated secondary antibody. FACS analysis was performed on a FACSCalibur cytometer (Becton Dickinson Immunocytometry Systems) and analyzed using FlowJo software (Tree Star Inc).

### Immunocytochemistry

Cells were fixed in 4% paraformaldehyde and stained with appropriate antibodies. If permeabilization was required, cells were treated with 0.1% saponin (BD Biosciences) prior to staining. Appropriate primary and fluorochrome-conjugated secondary antibodies were used. Cells were then counterstained with Hoechst 33342 (Invitrogen). The following antibodies were used: SSEA-3, TRA-1-60, OCT4, PAX6, p75, CD57 (BD biosciences), Nestin, TuJ1, MAP2, 04 (R&D System), Synapsin, TH, BRN3A, ISL1, P2X3R (Millipore), Glutamate, GABA, GFAP (Sigma), Nurr1 (Santa Cruz), vGluT1 (Abcam).

### Reverse Transcriptase PCR and Quantitative PCR (RT-PCR and RT-qPCR)

Total RNA purification was performed using RNeasy Mini Kit (Qiagen), including DNase I on-column digestion step, according to manufacturer's instructions. Purified RNA was quantified on a Nanodrop 2000 Spectrophotometer (Thermo Scientific). For RT-PCR, cDNA was synthesized from 500 ng of total RNA using iScript™ cDNA Synthesis Kit (BioRad). RT-PCR was performed using Recombinant Taq DNA Polymerase (Thermo Scientific). Random-primed Human Reference cDNA (Clontech) was used as a putative positive control. For RT-qPCR, cDNA was synthesized from 1 μg of total RNA using SuperScript III First-Strand Synthesis (Life Technologies). RT-qPCR was carried out using Platinum SYBR Green qPCR SuperMix-UDG (Life Technologies) utilizing manufacturer's recommended cycling conditions on an Mx3000P QPCR System (Stratagene). See Tables 1 and 2 for primers.

### Calcium Imaging

Differentiated cells at 1-2 weeks were loaded with Fluo-4-AM fluorescence dye (Invitrogen, Calif.) for 1 hr incubation followed by 45 mins period for de-esterification. Cells were washed and incubated in Hanks' balanced salt solution (HBSS), supplemented with 25 mM HEPES buffer, 5.5 mM Glucose. Calcium flux was monitored using an Olympus IX81 inverted epi-fluorescence microscope (Olympus, Markham, ON) coupled to a xenon arc lamp (EXFO, Quebec, QC). Indicated agonists, α,β-methylene-ATP or capsaicin, were diluted in the aforementioned solution and added to the well to give the final stimulation concentration (30 μM α,β-methylene-ATP, 1 μM capsaicin) using a dropping pipette and aspirator system. Fluorescence images were collected using an EMCCD camera (Photometrics, Tucson, Ark.) every 2 s through a GFP filter cube (Semrock, Rochester, N.Y.). In a subset of wells, ionomycin was added as a second stimulation for the dye loading control. For experiments using the selective P2X3 antagonist A-317491, the indicated concentration of compound was added to the wells 15 min before calcium imaging, and then calcium flux was measured as above. Off-line analysis of the intensity pattern of Fluo-4 signal was performed in ImageJ (NIH, Bethesda, Md.).

### Electrophysiology

Patch-clamp recordings were conducted at room temperature (˜21° C.) using an Axopatch 200B amplifier (Axon Instruments Inc., USA) from Cerebrasol (Montreal, Canada). Electrodes had a resistance of 2-4 MD when filled with recording solutions. The external recording solution contained 140 mM NaCl, 4.7 mM KCl, 1.2 mM MgCl₂, 2.5 mM CaCl₂, and 10 mM HEPES (pH 7.3), adjusted to 320 mOsm/l with glucose. Internal solutions. The intracellular solution contained 100 mM CsF, 45 mM CsCl, 10 mM NaCl, 5 mM EGTA, 1 mM MgCl₂, 10 mM HEPES (pH 7.3) adjusted to 300 mOsm with sucrose. For current-clamp recordings pipette solution of the following composition was used: 130 mM KCl, 0.5 mM EGTA, 10 mM HEPES, 1 mM MgCl₂, 5 mM Mg-ATP and 3 mM Na-GTP (pH 7.3), adjusted to 310 mOsm/l with glucose. Data were filtered at 1 KHz and digitized at 10 kHz. 25 mm cover slips with adhered cells were transferred to a recording chamber and cells visualized on an inverted Nikon microscope. Cells were continuously perfused at a slow perfusion rate of approximately 0.5 mL/min. For assessment of electrical excitability, experiments were conducted using the current-clamp recording configuration. Cells were held at approximately −60 mV and a series of hyperpolarizing and depolarizing current steps injected to characterize voltage gated currents and action potential initiation. For the assessment of voltage-activated sodium conductance (NaV), experiments were conducted using the voltage-clamp recording configuration. The presence of NaV conductance was determined using a simple step protocol from a holding potential (HP) of −120 mV to 0 mV for 30 ms, then back to −120 mV repeated at a frequency of 0.1 Hz.

### Gene Expression Analysis

Total RNA from hFib-iNSCOCT4 and hBD-iNPCOCT4 with or without SMAD/GSK-3 inhibitors was hybridized to Affymetrix Human Gene 1.0 ST arrays (London Regional Genomics Centre). Normalized expression data was applied to create hierarchical clustering and statistically significant gene lists (multiple test corrected p≤0.05, fold change≥1.5) using Partek Genomics Suite 6.6 (Partek Inc., St Louis, Mo., USA). For hierarchical clustering, primary human neural stem/progenitor cells were obtained from publicly available GEO source (GSE27505). Using Gene Set Enrichment Analysis software (Mootha et al., 2003; Subramanian et al., 2005), samples from hBD-iNPCOCT4 with or without inhibitors were compared to CD34-positive cord blood, and statistically significant (FDR q-value≤0.05) enriched gene set lists were generated. Tissue expression analysis was done using DAVID Bioinformatics database (Benjamini adjusted p≤0.01).

### Comparative Genomic Hybridization Array

Genomic DNA from samples was isolated using DNeasy kit (Qiagen) and concentrations were measured using NanoDrop. Sample DNA was hybridized to Agilent human CGH 4×44 k microarrays (Princess Margaret Genomics Centre, Toronto, ON). Standard human genomic DNA was hybridized to arrays as a reference. Partek Genomic Suite 6.6 software was used for analysis. Criteria of diploid copy number higher than 2.5 being as amplification and lower than 1.5 being as deletion was used, as well as statistical segmentation parameters with minimum genomic markers of 10 to specify genomic region and p-value threshold 0.001.

### Analysis of Catecholamines in Culture Media.

1 mL of culture medium was collected from culture wells. The oxidation status of the catecholamines was stabilized with 0.02 mL of an EGTA and glutathione buffer, and the sample was frozen at −30° C. Before analysis, the internal standard (3,4-Dihydroxybenzylamine) was added to the thawed medium for further processing using solid phase extraction cartridges as per manufacturer's recommendations (ChromSystems, Grafelfing, Germany). The samples were eluted into 0.12 mL and injected within 24 h in a High Performance Liquid Chromatographic System (HPLC, Waters 2695) coupled to an Electrochemical Detector (Waters 2465). The HPLC system used an analytical reverse phase column (Atlantis dC18; 5 micron; 4.6×150 mm; Waters) and an organic mobile phase (ChromSystems). Three physiological tyrosine-derived catecholamines (noradrenaline, adrenaline, dopamine) were used as standards. The concentration of catecholamines was calculated using the average area under the curve (n=3 injections) of the chromatograms of the calibration standards.

### In Vivo Transplantation

In vivo transplantation of cells into the neonatal mouse cortex has been described elsewhere (Zhu et al., 2014). Briefly, P2 to P4 old Nod.Scid (NOD.CB17-Prkdcscid/J) neonatal mice were injected with a total of 4×10⁵ BD NPC (2 injections into each right and left hemisphere, 1×10⁵ cells each site). Four weeks post injection, mice were sacrificed and brains were fixed. All mice were bred and maintained in the SCC-RI animal barrier facility at McMaster University. All animal procedures received the approval of the animal ethics board at McMaster University.

### Statistical Methods

Unless otherwise noted standard deviation was used in performing a student's t-test (two tailed) where *p=0.05 **p=0.01.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
qRT-PCR Primer List, Related to FIGS. 2, 4 and 5.

| | |
|---|---|
| GATA1 | F: 5′-GGGATCACACTGAGCTTGC (SEQ ID NO: 1) |
| | R: 5′-ACCCCTGATTCTGGTGTGG (SEQ ID NO: 2) |
| HOXB4 | F: 5′-CCTGGATGCGCAAAGTTCA (SEQ ID NO: 3) |
| | R: 5′-AATTCCTTCTCCAGCTCCAAGA (SEQ ID NO: 4) |
| PU.1 | F: 5′-ACGGATCTATACCAACGCCA (SEQ ID NO: 5) |
| | R: 5′-GGGGTGGAAGTCCCAGTAAT (SEQ ID NO: 6) |
| BMI1 | F: 5′-CAGAACAGATTGGATCGGAAA (SEQ ID NO: 7) |
| | R: 5′-CCGATCCAATCTGTTCTGGT (SEQ ID NO: 8) |
| BRN2 | F: 5′-AATAAGGCAAAAGGAAAGCAACT (SEQ ID NO: 9) |
| | R: 5′-CAAAACACATCATTACACCTGCT (SEQ ID NO: 10) |
| DCX1 | F: 5′-AGACCGGGGTTGTCAAAAAACTCTAC (SEQ ID NO: 11) |
| | R: 5′-TCAGGACCACAGGCAATAAACACATC (SEQ ID NO: 12) |
| ASCL1 | F: 5′-CAAGAGAGCGCAGCCTTAG (SEQ ID NO: 13) |
| | R: 5′-GCAAAAGTCAGTGCTGAACG (SEQ ID NO: 14) |
| HES1 | F: 5′-GAGCACAGAAAGTCATCAAAGC (SEQ ID NO: 15) |
| | R: 5′-TCCAGAATGTCCGCCTTC (SEQ ID NO: 16) |
| MYT1L | F: 5′-CAATGGAAAGGGATTTTAAGCA (SEQ ID NO: 17) |
| | R: 5′-TTTGAGATTATGTACCACGTTAGATG (SEQ ID NO: 18) |
| NESTIN | F: 5′-TCCAGGAACGGAAAATCAAG (SEQ ID NO: 19) |
| | R: 5′-GCCTCCTCATCCCCTACTTC (SEQ ID NO: 20) |
| NEUROD1 | F: 5′-GTTATTGTGTTGCCTTAGCACTTC (SEQ ID NO: 21) |
| | R: 5′-AGTGAAATGAATTGCTCAAATTGT (SEQ ID NO: 22) |
| NF68 | F: 5′-CAGACCGAAGTGGAGGAAAC (SEQ ID NO: 23) |
| | R: 5′-CCTCTTCCTTGTCCTTCTCCT (SEQ ID NO: 24) |
| NOTCH2 | F: 5′-ACATCATCACAGACTTGGTC (SEQ ID NO: 25) |
| | R: 5′-CATTATTGACAGCAGCTGCC (SEQ ID NO: 26) |
| PAX6 | F: 5′-CCGGCAGAAGATTGTAGAGC (SEQ ID NO: 27) |
| | R: 5′-CGTTGGACACGTTTTGATTG (SEQ ID NO: 28) |
| SOX1 | F: 5′-AACACTTGAAGCCCAGATGGA (SEQ ID NO: 29) |
| | R: 5′-GCAGGCTGAATTCGGTTCTC (SEQ ID NO: 30) |
| BRN3A | F: 5′-GTACCCGTCGCTGCACTC (SEQ ID NO: 31) |
| | R: 5′-GGCTTGAAAGGATGGCTCTTG (SEQ ID NO: 32) |
| ISL1 | F: 5′-TACAAAGTTACCAGCCACC (SEQ ID NO: 33) |
| | R: 5′-GGAAGTTGAGAGGACATTGA (SEQ ID NO: 34) |
| NTRK1 | F: 5′-TTGGCATGAGCAGGGATATCT (SEQ ID NO: 35) |
| | R: 5′-ACGGTACAGGATGCTCTCGG (SEQ ID NO: 36) |
| TAC1 | F: 5′-GCAGAAGAAATAGGAGCCAATG (SEQ ID NO: 37) |
| | R: 5-CGATTCTCTGCAGAAGATGCTC (SEQ ID NO: 38) |
| TRPV1 | F: 5′-GGCTGTCTTCATCATCCTGCTGCT (SEQ ID NO: 39) |
| | R: 5′-GTTCTTGCTCTCCTGTGCGATCTTGT (SEQ ID NO: 40) |
| P2RX3 | F: 5′-CCCCTCTTCAACTTTGAGAAGGGA (SEQ ID NO: 41) |
| | R: 5′-GTGAAGGAGTATTTGGGGATGCAC (SEQ ID NO: 42) |
| SCN9A | F: 5′-GCTCCGAGTCTTCAAGTTGG (SEQ ID NO: 43) |
| | R: 5′-GGTTGTTTGCATCAGGGTCT (SEQ ID NO: 44) |
| SCN10A | F: 5′-CAAATCTGAAACTGCTTCTGCCACA (SEQ ID NO: 45) |
| | R: 5′-CTAGGGCCCAGGGGCAATCAGCTCC (SEQ ID NO: 46) |
| SCN11A | F: 5′-CCCAGCAGCTGTTAAAGGAG (SEQ ID NO: 47) |
| | R: 5′-CTGGGACAGTCGTTTGGTTT (SEQ ID NO: 48) |
| TRPM8 | F: 5′-CAGCGCTGGAGGTGGATATTC (SEQ ID NO: 49) |
| | R: 5′-CACACACAGTGGCTTGGACTC (SEQ ID NO: 50) |

TABLE 2
RT-PCR Primers, Related to FIG. 8

| | |
|---|---|
| NTRK1 | F: GGCAGAGGTCTCTGTTCAGG (SEQ ID NO: 51) |
| | R: TGAACTCGAAAGGGTTGTCC (SEQ ID NO: 52) |
| NTRK2 | F: GTGGCGGAAAATCTTGTAGG (SEQ ID NO: 53) |
| | R: CCCCATTGTTCATGTGAGTG (SEQ ID NO: 54) |
| NTRK3 | F: CAACTGCAGCTGTGACATCC (SEQ ID NO: 55) |
| | R: GCCCAGTGACTATCCAGTCC (SEQ ID NO: 56) |
| NEFH | F: GGTGAACACAGACGCTATGC (SEQ ID NO: 57) |
| | R: TCTCCCACTTGGTGTTCCTC (SEQ ID NO: 58) |
| CALCA | F: TGCACTGGTGCAGGACTATG (SEQ ID NO: 59) |
| | R: AAGGCTTTGGAACCCACATT (SEQ ID NO: 60) |
| GUSB | F: ACGACACCCACCACCTACAT (SEQ ID NO: 61) |
| | R: TACAGATAGGCAGGGCGTTC (SEQ ID NO: 62) |
| TBP | F: GAACCACGGCACTGATTTTC (SEQ ID NO: 63) |
| | R: CACAGCTCCCCACCATATTC (SEQ ID NO: 64) |

TABLE 3
Structures of Inhibitors

| Inhibitor | Structure |
|---|---|
| SB431542 | 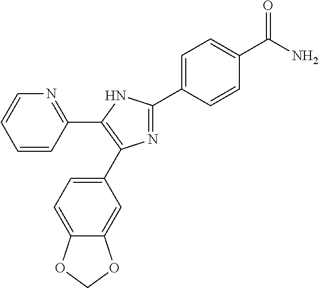 [SMILES: NC(=O)c1ccc(-c2nc(-c3ccc4c(c3)OCO4)c(-c3ccccn3)[nH]2)cc1] |
| LDN-193189 | 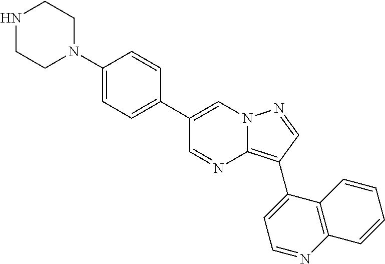 [SMILES: c1ccc2c(-c3cnn4cc(-c5ccc(N6CCNCC6)cc5)cnc34)ccnc2c1] |
| CHIR99021 | 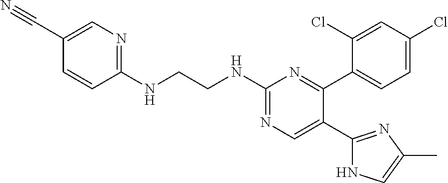 [SMILES: Cc1c[nH]c(-c2cnc(NCCNc3ccc(C#N)cn3)nc2-c2ccc(Cl)cc2Cl)n1] |

### REFERENCES

Barberi, T., Klivenyi, P., Calingasan, N. Y., Lee, H., Kawamata, H., Loonam, K., Perrier, A. L., Bruses, J., Rubio, M. E., Topf, N., et al. (2003). Neural subtype specification of fertilization and nuclear transfer embryonic stem cells and application in parkinsonian mice. Nat Biotechnol 21, 1200-1207.
Bennett, D. L., and Woods, C. G. (2014). Painful and painless channelopathies. The Lancet Neurology 13, 587-599.
Blanchard, J. W., Eade, K. T., Szucs, A., Lo Sardo, V., Tsunemoto, R. K., Williams, D., Sanna, P. P., and Baldwin, K. K. (2015). Selective conversion of fibroblasts into peripheral sensory neurons. Nature neuroscience 18, 25-35.
Boyette-Davis, J., Xin, W., Zhang, H., and Dougherty, P. M. (2011). Intraepidermal nerve fiber loss corresponds to the development of taxol-induced hyperalgesia and can be prevented by treatment with minocycline. Pain 152, 308-313.
Broxmeyer, H. E. (2010). Will iPS cells enhance therapeutic applicability of cord blood cells and banking? Cell Stem Cell 6, 21-24.
Castano, J., Menendez, P., Bruzos-Cidon, C., Straccia, M., Sousa, A., Zabaleta, L., Vazquez, N., Zubiarrain, A., Sonntag, K. C., Ugedo, L., et al. (2014). Fast and efficient neural conversion of human hematopoietic cells. Stem cell reports 3, 1118-1131.
Caterina, M. J., Schumacher, M. A., Tominaga, M., Rosen, T. A., Levine, J. D., and Julius, D. (1997). The capsaicin receptor: a heat-activated ion channel in the pain pathway. Nature 389, 816-824.
Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nat Biotechnol 27, 275-280.
Chambers, S. M., Qi, Y., Mica, Y., Lee, G., Zhang X. J., Niu, L., Bilsland, J., Cao, L., Stevens, E., Whiting, P., et al. (2012). Combined small-molecule inhibition accelerates developmental timing and converts human pluripotent stem cells into nociceptors. Nat Biotechnol 30, 715-720.
Cunningham, J. J., Ulbright, T. M., Pera, M. F., and Looijenga, L. H. (2012). Lessons from human teratomas to guide development of safe stem cell therapies. Nat Biotechnol 30, 849-857.
Efe, J. A., Hilcove, S., Kim, J., Zhou, H., Ouyang, K., Wang, G., Chen, J., and Ding, S. (2011). Conversion of mouse fibroblasts into cardiomyocytes using a direct reprogramming strategy. Nat Cell Biol 13, 215-222.
Guo, X., Spradling, S., Stancescu, M., Lambert, S., and Hickman, J. J. (2013). Derivation of sensory neurons and neural crest stem cells from human neural progenitor hNP1. Biomaterials 34, 4418-4427.
http://brainbank.ucla.edu The Human Brain and Spinal Fluid Resource Center (HBSFRC).
http://www.clsa-elcv.ca/ Canadian Longitudinal Study on Aging (clsa).
Jarvis, M. F., Burgard, E. C., McGaraughty, S., Honore, P., Lynch, K., Brennan, T. J., Subieta, A., Van Biesen, T., Cartmell, J., Bianchi, B., et al. (2002). A-317491, a novel potent and selective non-nucleotide antagonist of P2X3 and P2X2/3 receptors, reduces chronic inflammatory and neuropathic pain in the rat. Proceedings of the National Academy of Sciences of the United States of America 99, 17179-17184.
Kim, Y. J., Lim, H., Li, Z., Oh, Y., Kovlyagina, I., Choi, I. Y., Dong, X., and Lee, G. (2014). Generation of multipotent induced neural crest by direct reprogramming of human postnatal fibroblasts with a single transcription factor. Cell Stem Cell 15, 497-506.
Kriks, S., Shim, J. W., Piao, J., Ganat, Y. M., Wakeman, D. R., Xie, Z., Carrillo-Reid, L., Auyeung, G., Antonacci, C., Buch, A., et al. (2011). Dopamine neurons derived from human ES cells efficiently engraft in animal models of Parkinson's disease. Nature 480, 547-551.
Lee, J. H., Lee, J. B., Shapovalova, Z., Fiebig-Comyn, A., Mitchell, R. R., Laronde, S., Szabo, E., Benoit, Y. D., and Bhatia, M. (2014). Somatic transcriptome priming gates lineage-specific differentiation potential of human-induced pluripotent stem cell states. Nature communications 5, 5605.
Lee, K. S., Zhou, W., Scott-McKean, J. J., Emmerling, K. L., Cai, G. Y., Krah, D. L., Costa, A. C., Freed, C. R., and Levin, M. J. (2012). Human sensory neurons derived from induced pluripotent stem cells support varicella-zoster virus infection. PLoS One 7, e53010.
Li, W., Sun, W., Zhang, Y., Wei, W., Ambasudhan, R., Xia, P., Talantova, M., Lin, T., Kim, J., Wang, X., et al. (2011). Rapid induction and long-term self-renewal of primitive neural precursors from human embryonic stem cells by small molecule inhibitors. Proceedings of the National Academy of Sciences of the United States of America 108, 8299-8304.
Lujan, E., Chanda, S., Ahlenius, H., Sudhof, T. C., and Wernig, M. (2012). Direct conversion of mouse fibroblasts to self-renewing, tripotent neural precursor cells. Proceedings of the National Academy of Sciences of the United States of America 109, 2527-2532.
Ma, L., Hu, B., Liu, Y., Vermilyea, S. C., Liu, H., Gao, L., Sun, Y., Zhang, X., and Zhang, S. C. (2012). Human embryonic stem cell-derived GABA neurons correct locomotion deficits in quinolinic acid-lesioned mice. Cell Stem Cell 10, 455-464.
Mitchell, R., Szabo, E., Shapovalova, Z., Aslostovar, L., Makondo, K., and Bhatia, M. (2014a). Molecular evidence for OCT4-induced plasticity in adult human fibroblasts required for direct cell fate conversion to lineage specific progenitors. Stem cells 32, 2178-2187.
Mitchell, R. R., Szabo, E., Benoit, Y. D., Case, D. T., Mechael, R., Alamilla, J., Lee, J. H., Fiebig-Comyn, A., Gillespie, D. C., and Bhatia, M. (2014b). Activation of Neural Cell Fate Programs Toward Direct Conversion of Adult Human Fibroblasts into Tri-Potent Neural Progenitors Using OCT-4. Stem Cells Dev 23, 1937-1946.
Najm, F. J., Lager, A. M., Zaremba, A., Wyatt, K., Caprariello, A. V., Factor, D. C., Karl, R. T., Maeda, T., Miller, R. H., and Tesar, P. J. (2013). Transcription factor-mediated reprogramming of fibroblasts to expandable, myelinogenic oligodendrocyte progenitor cells. Nat Biotechnol 31, 426-433.
Pang, Z. P., Yang, N., Vierbuchen, T., Ostermeier, A., Fuentes, D. R., Yang, T. Q., Citri, A., Sebastiano, V., Marro, S., Sudhof, T. C., et al. (2011). Induction of human neuronal cells by defined transcription factors. Nature 476, 220-223.
Pino, B. M. d. (2010a). Chemotherapy-induced peripheral neuropathy, N. C. Institute, ed., pp. 6.
Pino, B. M. d. (2010b). Chemotherapy-induced peripheral neuropathy. In NCI Cancer Bulletin (National Cancer Institute).
Rieske, P., Krynska, B., and Azizi, S. A. (2005). Human fibroblast-derived cell lines have characteristics of embryonic stem cells and cells of neuro-ectodermal origin. Differentiation; research in biological diversity 73, 474-483.
Ring, K. L., Tong, L. M., Balestra, M. E., Javier, R., Andrews-Zwilling, Y., Li, G., Walker, D., Zhang, W. R., Kreitzer, A. C., and Huang, Y. (2012). Direct reprogramming of mouse and human fibroblasts into multipotent neural stem cells with a single factor. Cell Stem Cell 11, 100-109.
Sancho-Martinez, I., Baek, S. H., and Izpisua Belmonte, J. C. (2012). Lineage conversion methodologies meet the reprogramming toolbox. Nat Cell Biol 14, 892-899.
Stacey, G. N., Crook, J. M., Hei, D., and Ludwig, T. (2013). Banking human induced pluripotent stem cells: lessons learned from embryonic stem cells? Cell Stem Cell 13, 385-388.
Szabo, E., Rampalli, S., Risueno, R. M., Schnerch, A., Mitchell, R., Fiebig-Comyn, A., Levadoux-Martin, M., and Bhatia, M. (2010). Direct conversion of human fibroblasts to multilineage blood progenitors. Nature 468, 521-526.
Takahashi, K., and Yamanaka, S. (2006). Induction of pluripotent stem cells from mouse embryonic and adult fibroblast cultures by defined factors. Cell 126, 663-676.
Tesfaye, S., Boulton, A. J., and Dickenson, A. H. (2013). Mechanisms and management of diabetic painful distal symmetrical polyneuropathy. Diabetes Care 36, 2456-2465.
Wainger, B. J., Buttermore, E. D., Oliveira, J. T., Mellin, C., Lee, S., Saber, W. A., Wang, A. J., Ichida, J. K., Chiu, I. M., Barrett, L., et al. (2015). Modeling pain in vitro using nociceptor neurons reprogrammed from fibroblasts. Nature neuroscience 18, 17-24.
Wapinski, O. L., Vierbuchen, T., Qu, K., Lee, Q. Y., Chanda, S., Fuentes, D. R., Giresi, P. G., Ng, Y. H., Marro, S., Neff, N. F., et al. (2013). Hierarchical mechanisms for direct reprogramming of fibroblasts to neurons. Cell 155, 621-635.
Yu, K. R., Shin, J. H., Kim, J. J., Koog, M. G., Lee, J. Y., Choi, S. W., Kim, H. S., Seo, Y., Lee, S., Shin, T. H., et al. (2015). Rapid and Efficient Direct Conversion of Human Adult Somatic Cells into Neural Stem Cells by HMGA2/let-7b. Cell Rep.
Zhu, S., Ambasudhan, R., Sun, W., Kim, H. J., Talantova, M., Wang, X., Zhang, M., Zhang, Y., Laurent, T., Parker, J., et al. (2014). Small molecules enable OCT4-mediated direct reprogramming into expandable human neural stem cells. Cell Res 24, 126-129.

1. A method of generating induced neural progenitor cells from CD34⁺/CD45⁺ blood cells comprising:
a) providing CD34⁺/CD45⁺ blood cells that ectopically express, overexpress or are treated with a POU domain containing gene or protein and culturing said cells in media to allow expression of the POU domain containing gene or protein; and
b) culturing the cells produced in (a) in basal neural progenitor media supplemented with inhibitors of Smad and GSK-3β to produce induced neural progenitor cells;
wherein induced neural progenitor cells are generated without traversing the pluripotent state.

2. The method of claim 1, wherein the cells in (a) are cultured in hematopoietic stem cell culture media, optionally for 2-4 days, followed by reprogramming media, optionally for 4-7 days.

3. The method of claim 1, wherein the method further comprises maintaining or expanding the cells produced in (b) in neural induction media.

4. The method of claim 1, wherein CD34⁺/CD45⁺ blood cells that ectopically express a POU domain containing gene or protein in (a) are produced by lentiviral transduction or are produced by providing an exogenous POU domain containing gene or protein.

5. (canceled)

6. The method of claim 1, wherein the POU domain containing gene or protein is an Oct gene or protein, wherein the Oct gene or protein is Oct-4, -2, -1 or -11.

7. (canceled)

8. The method of claim 6, wherein the Oct gene or protein is Oct-4.

9. The method of claim 1, wherein the CD34⁺/CD45⁺ blood cells are derived from peripheral blood or umbilical cord blood.

10. (canceled)

11. (canceled)

12. The method of claim 2, wherein the hematopoietic stem cell culture media comprises SCF, Fit-3L, IL-3 and TPO.

13. (canceled)

14. (canceled)

15. The method of claim 2, wherein the reprogramming media comprises DMEM/F12, 20% Knockout Serum Replacement and bFGF.

16. The method of claim 1, wherein the inhibitor of Smad is SB431542, LDN-193189, and/or Noggin.

17. The method of claim 1, wherein the inhibitor of GSK-3β is CHIR99021.

18. (canceled)

19. (canceled)

20. The method of claim 1, further comprising culturing the cells in differentiation medium under conditions that allow production of differentiated cells.

21. (canceled)

22. The method of claim 24, wherein the differentiated cells are GABA neurons, DA neurons, sensory neurons, astrocytes or oligodendrocytes.

23.-28. (canceled)

29. A method of screening progenitor or cells derived therefrom comprising:
a) preparing a culture of progenitor or differentiated cells by the method of claim 1;
b) treating the cells with a test agent or agents; and
c) subjecting the cells to analysis.

30. A method of screening for a compound that modulates the activity, function, viability and/or morphology of sensory neurons comprising:
a) preparing a culture of sensory neurons by the method of claim 22;
b) treating the cells with a test compound; and
c) testing the cells for a compound that modulates the activity, function, viability and/or morphology compared to a control in the absence of test compound.

31. The method of claim 30, wherein the test compound is screened for the effect of decreasing or increasing viability of sensory neuron cells, the effect of decreasing or increasing neurite length of the sensory neuron cells compared to control.

32. (canceled)

33. The method of claim 31, wherein identification of a test compound as capable of increasing viability or neurite length indicates that the compound is a candidate for treating neuropathies, such as diabetic-induced neuropathy.

34. (canceled)

35. (canceled)

36. The method of claim 30, wherein the test compound is screened in the presence of a chemotherapeutic agent that is known to cause neuropathy and the effect of the test compound in alleviating the neuropathy compared to control is measured.

37. (canceled)
